(19) United States
(12) Patent Application Publication
(10) Pub. No.: US 2011/0045045 A1
(43) Pub. Date: Feb. 24, 2011

(54) Production of and uses for decellularized lung tissue

(76) Inventors: Joaquin Cortiella, Galveston, TX (US); Joan E. Nichols, Galveston, TX (US); Jean A. Niles, Galveston, TX (US)

Correspondence Address: Dr. Benjamin Adler;ADLER & ASSOCIATES, 8011 Candle Lane, Houston, TX, 77071, US

(21) Appl. No.: 12/803,774

(22) Filed: Jul. 6, 2010

Related U.S. Application Data
(60) Provisional application No. 61/270,348, filed on Jul. 7, 2009.

(51) Int. Cl. A61K 9/00; C12N 5/02; C12N 5/071; A61K 35/42; A61P 11/00

(52) U.S. Cl. 424/422; 435/395; 435/377; 424/93.7

(57) ABSTRACT

The present invention provides a process of producing a decellularized extracellular matrix DC lung from native lung tissue using rapid freeze/thaw cycling to induce cellular damage and the constant circulation of a detergent or peracetic acid and enzymatic digestion with DNAase/RNAase within a continuously rotating bioreactor. Also, provided are methods to produce a functional engineered lung tissue on the DC lung using endogenous lung progenitor cells. In addition, a composition comprising the DC lung and the endogenous lung progenitor cells seeded therein or thereon and an implantable composition comprising the functional engineered lung tissue which are useful in methods of treating a lung to restore at least some function thereto.

### CROSS-REFERENCE TO RELATED APPLICATION

This nonprovisional application claims benefit of priority under 35 U.S.C. §119(e) of provisional U.S. Ser. No. 61/270,348, filed Jul. 7, 2009, now abandoned, the entirety of which is hereby incorporated by reference.

### BACKGROUND OF THE INVENTION

1. Field of the Invention

The present invention relates to the fields of engineered biomaterials and regenerative medicine. More specifically, the present invention provides a process for lung tissue decellularization and methods for producing engineered functional lung tissue and for treating pulmonary diseases or disorders, etc. using the functional engineered lung tissue.

2. Description of the Related Art

Diseases of the lung, including chronic obstructive pulmonary disorders (COPD), are collectively the fourth leading cause of death in the world (1). In 2005, approximately 1 in 20 deaths in the U.S. had COPD as the underlying cause (2). In those patients suffering from severe lung disease standard medical care is palliative with organ transplant surgery as the only option. One of the major problems with lung transplantation as a therapy is the lack of available organs and unfortunately many patients die from the associated complications of the disease before receiving an appropriately tissue matched organ. For the majority of those individuals suffering from lung disease the cost of COPD poses a major economic burden to the U.S. For instance, the National Heart, Lung, and Blood Institute (NHLBI) provides the single current estimate of total (direct and indirect) annual cost of COPD to the U.S., as $38.8 billion in 2005 dollars (3). Making COPD-associated healthcare utilization and expenditures a considerable cost burden to the U.S. health care system.

The generation of new lung tissue through tissue engineering offers the possibility of developing novel treatments for lung diseases/disorders and may provide an answer for the current organ shortage. An understanding of the matrix conditions and ECM components that lend support to lung development and repair is an important area of research that could be translated into the development of engineered tissues worthy of clinical application.

Tissue engineering for regenerative medicine purposes should encompass the reconstruction of tissue equivalents to replace the physiologic functions of tissues lost due to disease or injury. Clearly engineering of a complex organ such as lung presents so many scientific challenges that development of clinically applicable replacement tissues has not yet been realized. In order to develop an engineered lung appropriate scaffolding or matrix material must first be developed to provide the framework which is necessary to support cell growth and tissue development in a way that it does not impede the elasticity of the engineered tissue or affect the different functional areas of the lung. Both synthetic and natural polymers have been studied for use in lung tissue engineering (4-8).

Synthetic material such as polyglycolic acid (PGA) has been used to produce lung tissue in vitro but in vivo implantation of endogenous lung stem cell/PGA constructs did not support lung tissue growth (5) due to a foreign body response created by the PGA. Natural materials that have been used to engineer lung tissue include collagen (4, 6, 8), Matrigel (6) and Gelfoam (7). In vivo use of these natural scaffolds supports tissue growth although development of lung tissue using these materials has not been substantial (6, 7). Additionally, all of these studies utilized simple matrices which were not designed to meet the requirements for lung in terms of matrix composition, elasticity or porosity (8, 9).

To meet the needs of the lung, matrix material used for engineering lung tissue should mimic the natural design of the structural material that forms native lung and have a highly organized three dimensional (3D) structure with shape and pore size similar to that found in the broncho/alveolar regions of the lung. The material must also possess a sufficiently large surface area for cell/ECM attachment, cell migration, transport of nutrients and transport of waste materials. Recently there was a successful attempt at repopulating a decellularized heart with cardiac and endothelial cells (10). The natural scaffold was instrumental in supporting cellular growth and maintaining the hearts function after repopulation.

The best matrix to engineer a lung might be to use its own cytoskeleton or extracellular matrix (ECM). The assumption would be that tensile strength, pore size and the geometry of a decellularized (DC) lung would be preserved while also accommodating the metabolic and functional demands of the cells specific to the organ. Especially since the lung is a complex organ with many different cell types located in different parts of the lung which are often involved in providing different functions.

There are however some limitations to be considered before using a decellularized or natural extracellular matrix. The effects of the decellularization process on the matrix and the mechanical integrity of the ECM must be considered. It has been shown that matrix can be weakened or degraded by the decellularization process which in turn may interfere with later cell migration and differentiation (11). Recently a tissue-engineered trachea formed from a decellularized cadaveric trachea which was recellularized with the patient's own mesenchymal stem cells and epithelial cells was transplanted successfully into a patient with bronchial stenosis (12). Although a number of protocols exist in the current literature to decellularize heart (10), trachea (11) or other tissues, no protocol to decellularize lung currently exists.

The prior art is deficient in processes and methods to produce a matrix from native tissue on which to engineer tissue. More specifically, the prior art is deficient in processes and methods to produce a native decellularized tissue extra cellular matrix and to culture or bioengineer the tissue onto the decellularized matrix and in the compositions so formed. The present invention fulfills this long-standing need and desire in the art.

### SUMMARY OF THE INVENTION

The present invention is directed a process for producing decellularized lung extracellular matrix (DC lung). The process comprises inducing cellular damage to native lung tissue and removing cellular debris produced by the cellular damage to the lung tissue, wherein remaining tissue is the decellularized lung extracellular matrix. The present invention is also directed to a related process further comprising seeding onto or into the DC lung ECM endogenous progenitor lung cells to produce a cell:matrix construct. The present invention further is directed to another related process further comprising culturing the cell:matrix construct in vitro in a bioreactor under conditions effective to induce differentiation and growth of the cells toward a lung lineage within the matrix thereby producing functional lung tissue. The present invention is directed to another related process further comprising implanting the functional lung tissue at one or more non-functioning sites of interest within a lung to restore at least some function thereto.

The present invention also is directed to a method for producing engineered functional three-dimensional lung tissue. The method comprises decellularizing native lung tissue to produce a decellularized lung extracellular matrix (DC lung). Endogenous progenitor lung cells are isolated and seeded onto or into the DC lung to produce a cell:matrix construct, The cell:matrix construct is cultured in a bioreactor under conditions effective to induce differentiation and growth of the cells toward a lung lineage within the matrix thereby producing the engineered functional three-dimensional lung tissue. The present invention is directed to a related method further comprising implanting the engineered lung tissue into a subject having a pulmonary disease, a pulmonary disorder or an injury to pulmonary tissue.

The present invention is directed further to a composition comprising decellularized lung extracellular matrix and endogenous lung progenitor cells seeded thereon or therein. The present invention is directed further still to an implantable composition comprising decellularized lung extracellular matrix and engineered functional lung tissue differentiated from endogenous lung progenitor cells grown in or on the decellularized extracellular matrix. The present invention is directed further still to a method for treating a lung to restore function thereto in a subject in need of such treatment. The method comprises implanting into the lung of the subject the implantable composition described herein where growth of the lung tissue comprising the implantable composition restores at least partial function to the lung.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is noted, however, that the appended drawings illustrate preferred embodiments of the invention and therefore are not to be considered limiting in their scope.

FIG. 1 is a flow chart depicting the decellularization protocol including physical (freezing), mechanical (rotation of bioreactor with circulation of detergent) and enzymatic (DNAase and RNAase treatment) steps necessary to produce rat MHC-1, DNA, RNA free DC lung tissue.

FIGS. 2A-2L depict evaluation of decellularization process. (FIG. 2A) Appearance of rat trachea with attached lungs immediately after excision. (FIG. 2B) Condition of whole rat lungs after freeze-thawing followed by treatment in 1% SDS for 1 week in a 50 ml bioreactor chamber. (FIG. 2C) Gross condition of whole lung after 1% SDS treatment for 5 weeks. Lung is shown in the rotary bioreactor chamber during the final antibiotic/antimycotic wash. (FIGS. 2D-2G) Confocal images of 7 um frozen-sections of whole lung (FIG. 2D) stained for the presence of cell membrane associated rat MHC-I after 1 week in 1% SDS treatment show extensive regions positive for cell debris. DAPI staining for nuclei/nuclear material also showed the presence of many intact nuclei. Staining with human MHC-1 was used as a negative control. Magnification 400×. (FIG. 2E) Examination of 7 um tissue sections after 4 weeks of 1% SDS treatment demonstrated that a few areas remained positive for rat MHC-1 or DAPI. Arrow points to low level staining in tissue section. Staining with human MHC-1 was used as a negative control. Magnifications 400×. PI staining of tissue sections (FIG. 2F) prior to and (FIG. 2G) after DNAase/RNAase treatment indicated that significant loss of nuclear material had occurred. White arrows point to PI positive regions. Magnifications 400×. (FIG. 2H) Gel electrophoresis was used to evaluate the DNA content within AC rat lung after 5 weeks of 1% SDS treatment with (−) and without (+) DNAase treatment. Gels indicated that trace amounts of DNA remained even after DNAase treatment of the lung ECM matrix. (FIG. 2I) At the end of the decellularization process AC lung was uniformly clear and glassy in appearance. (FIG. 2J-2L) 4 um frozen sections of AC rat lung matrix were photographed using transmitted white light using a Zeiss LSM 510 Meta inverted microscope to show the fibrilar network of the remaining ECM after successful decellularization of the tissues. These sections showed the AC lung ECM substructure in the regions corresponding to (FIG. 2J) distal lung and (FIG. 2K-2L) upper airway. Regions near (FIG. 2K) the main bronchus near the carina and (FIG. 2L) the trachea show the dense fibrous nature of upper lung. Magnifications 400×. Abbreviations: 4′,6-diamidino-2-phenylindole, dihydrochloride, DAPI; major histocompatibility molecule, MHC; human, H; rat, R.

FIGS. 3A-3E described examination of Gross Structure of DC lung Matrix. (FIG. 3A) Image of intact DC rat lung showing underlying substructure formed by remnants of bronchi and branching airway ECM. (FIG. 3B and FIG. 3C) Confocal images of 7 um frozen sections of DC lung stained for presence of (FIG. 3B) collagen-I (green) or (FIG. 3C) elastin (green). Magnifications 630×, bar in each is 10 um. (D) Two photon images of DC rat lung viewed to a depth of 180 um (field of view is 320 um). SHG microscopy was used to visualize fibrilar collagen (red) in this 3D reconstruction, autofluorescence of cells, elastin and other ECM (green). (FIG. 3E) XY single plane two-photon imaging of DC rat lung autofluorescence was done at depths of 27, 38, 51, 86, 120 and 179 um (top and bottom set of three images). In the middle three images, SHG microscopy was used to visualize fibrillar collagen (red) at depths of 27, 38 and 51 um.

FIGS. 4A-4P are images comparing biocompatible matrices with and without progenitor cells. 4 um sections of hydrogel-type I collagen matrix (FIG. 4A), Matrigel (FIG. 4B) or Gelfoam (FIG. 4C) showing sub-structure of each matrix material. Murine embryonic stem cells (mESCs) were seeded and then cultured for 1 week on DC lung (FIG. 4D), hydrogel-type I collagen matrix (FIG. 4E), Matrigel (FIG. 4F) or Gelfoam (FIG. 4G) were stained to indicate the position of nuclei using DAPI. FIG. 4H characterizes the heterogenous mixture of SSEA-4+, Oct-4+ human endogenous lung progenitor cells (ELPCs). FIGS. 4I-4J show 7 um sections of normal human lung stained to indicate the position of cell nuclei using DAPI. Endogenous lung progenitor cells were seeded and then cultured for 1 week (FIGS. 4K-4N) or 3 weeks (FIGS. 4O-4P). 7 um sections of DC lung (FIG. 4K), hydrogel-type I collagen matrix (FIG. 4L), Matrigel (FIG. 4M) or Gelfoam (FIG. 4N) or DC lung (FIGS. 4O-4P) after 3 weeks of culture were stained to indicate the position of nuclei using DAPI. Magnification is 400×.

FIGS. 5A-5T are micrographs illustrating architecture and protein content in rat lung tissue and decellularized extracellular matrix. 2-photon microscopy was used to examine the architecture and collagen content of normal rat lung (FIG. 5A) showing collagen (green) and cells (red), DC lung (FIG. 5B) showing collagen (red) and remaining ECM (green or DC lung (b) showing collagen (red) and mESCs as well as remaining ECM (green). Confocal image of 7 um frozen sections showing positive staining for Mouse MHC-1 (red) and negative staining for rat MHC-1 (green) after 2 weeks of culture (FIG. 5D). DAPI nuclear staining (blue). Examination of collagen (red) (FIG. 5E), laminin (red) (FIG. 5F), collagen (red) and cytokeratin expression (FIG. 5G), elastin (green) (FIG. 5H), cytokeratin (red) (b), Pro-SPC (green) (FIG. 5J), CC10 (red) and cytokeratin (green) (FIG. 5K), CD140a (red) (FIG. 5L), by DC lung seeded with mESCs and cultured for 2 weeks. DAPI nuclear stain (blue). Antibody control for secondary antibodies used in FIGS. 5E-5L (FIG. 5M). Expression of Aquaporin-5 (yellow) and cytokeratin (red) by mESCs on DC lung matrix (FIGS. 5N-5O) and CD31 (FIG. 5P) after 3 weeks of culture. Antibody control for secondary antibodies used in FIGS. 5N-5S (FIG. 5Q). Expression of cytokeratin in ECM regions that were originally trachea (b), CD31 (red) and cytokeratin (green) (FIG. 5S) and in regions of ECM that were near the carina showing expression of CC10 (red) cytokeratin (green) and TT-F1 (yellow) (FIG. 5T) after 4 weeks of culture. DAPI nuclear stain (blue) (FIGS. 5D-5T). Magnification 200× (FIGS. 5A-5D and 5S), 400× (FIGS. 5E-5N, 5P-5R and 5T) and 630× (FIG. 5O).

FIGS. 6A-6M show 4 um sections of (FIG. 6A) DC rat lung, (FIG. 6C) Matrigel, (FIG. 6E) Gelfoam or (FIG. 6G) Collagen-UPF-127 hydrogel matrix were photographed using transmitted white light on a Zeiss LSM 510 Meta inverted microscope to show the substructure of each matrix material. Magnifications for A-H, bar equals 20 um. MESC repopulation of DC rat lung was compared to repopulation of Matrigel, Gelfoam and collagen-I/PF-127 hydrogel matrices. After seeding with mESCs each 0.5 cm3 piece of matrix material was cultured for 7 days (FIG. 6B, FIG. 6D, FIG. 6F, and FIG. 6H). 7 um frozen sections of each matrix were stained with DAPI to allow for the visualization of cell nuclei. Visual inspection using confocal microscopy indicated that more cells were found in (FIG. 6B) DC lung when compared to (FIG. 6D) Matrigel, (FIG. 6F) Gelfoam or (FIG. 6H) collagen-I/PF-127 hydrogel matrix. Magnifications of each (FIGS. 6E-6H) were 400×. (FIG. 6I) After 7 days of culture cells were isolated from each matrix and the number of viable cells was determined using a live/dead cell viability assay (Molecular Probes) with analysis by flow cytometry. Significantly more viable cells were recovered from the DC lung matrix than the other matrices examined (P<0.001). Cells isolated from each matrix were also examined for level of (FIG. 6J) apoptosis (TUNEL evaluation) using flow cytometry. Significantly more cells from plate compared to 3D-matrix culture were TUNEL positive (P<0.001). DC lung also had less TUNEL positive (P<0.05) cells when compared to Matrigel and collagen-UPF-127 but not when compared to Gelfoam. (FIGS. 6K-6M) Flow cytometric examination of the cells isolated from each matrix were stained for the presence of (FIG. 6K) cytokeratin, (FIG. 6L) CD31 and (FIG. 6M) pro-SPC in order to examine the influence of cell matrix on mESC differentiation. (FIG. 6K) Significantly more cells from DC lung matrices were positive for cytokeratin (P<0.001) compared to Matrigel or Gelfoam (P<0.01 for DC lung compared to collagen-I/PF127), (FIG. 6L) CD31 (P<0.001 for DC lung compared to all matrices used) and (FIG. 6M) pro-SPC (P<0.001 for DC lung compared to all matrices used). (*) indicates P<0.001 for DC lung matrix compared to the all other matrices used and (∞) indicates P<0.05. Abbreviations: Plate culture, PC; acellular lung, AC; Matrigel, MG; Gelfoam, GEF; collagen-UPF-127, C-1; growth factors, GF; pluronic F-127, PF-127; pro-surfactant protein C, PRO-SPC

FIGS. 7A-7M are drawn to recellularized rat lung after 14 days of culture (FIG. 7A) Gross image of DC rat lung (left) next to mESC-recellularized lung (right) after culture for 14 days showing contraction of the ECM. (FIGS. 7B-7D) Two-photon imaging, 3D reconstructions of (FIG. 7B) Normal fresh rat lung tissue, (FIG. 7C) AC lung and (FIG. 7D) Recellularized rat lung tissue. Green color corresponds to SHG showing collagen and red to autofluorescence of cells, elastin and other ECM. (FIG. 7D) Recellularized lung tissue imaged at a depth of 22 um (FIG. 7E) Confocal image of 7 um frozen section of AC lung recellularized with mESC after 14 days of culture stained for expression of collagen-I (green) and elastin (red). Magnifications 630×. (FIG. 7F) Control for FIG. G stained with secondary antibody donkey anti-goat IgG conjugated to Alexa Fluor 680 and (FIG. 7G) sections stained for laminin, bar 20 um. (FIG. 7H) Control for FIG. I (bar 20 um) stained with secondary antibody rabbit anti-mouse IgG conjugated to Alexa Fluor 488 followed by donkey anti-goat conjugated to Alexa Fluor 680 and (FIG. 7I) sections stained for cytokeratin-18 (green) and collagen-IV (red). Magnifications of (FIGS. 7E-7I) 630×. (FIG. 7J) Control for FIG. K (bar 20 um) stained with IgG isotype antibody conjugated to Pe-CY5 and evaluation of (FIG. 7K) CD140a (PDGFR-α) expression (red). (FIG. 7L) Control for FIG. M stained with rabbit anti-mouse IgG conjugated to Alexa Fluor 488 and (FIG. 7M) evaluation of expression of pro-SPC (red) by type II pneumocytes. Magnifications for E-M were 400×. (E-M) Tissues were counterstained with DAPI to view nuclei (blue). Abbreviations: platelet derived growth factor receptor-alpha (PDGFR-α); pro surfactant protein C (pro-SPC).

FIGS. 8A-8V are micrographs illustrating expression of proteins in normal human lung tissue and rat DC lung seeded with human endogenous lung progenitor cells. Normal human lung stained for expression of cytokeratin (green) and CC10 in trachea (FIG. 8A), Pro-SPC (red) and cytokeratin (green) (FIG. 8B) in upper region of lung near the carina and CD31 (red) and cytokeratin (green) and DAPI (nuclei, blue) (b). Confocal image of 7 um frozen sections of rat DC lung seeded with human endogenous lung progenitor cells which were cultured for 2 weeks and then stained with DAPI to show presence of nuclei (FIG. 8D). Examination of human MHC-1 (red) (FIG. 8E), CD140a (red) (b), Pro-SPC (red) (FIG. 8G), cytokeratin (red) (FIG. 8H), aquaporin-5 (lavender) and cytokeratin (red) (FIG. 8I), alpha-actin (red) (FIG. 8J) in these cultures of human endogenous lung progenitor cells on rat DC lung. Evaluation of presence of nuclei of cells in ECM regions that were originally trachea (FIG. 8K) in 2 week cultures using DAPI nuclear stain (blue) as well as expression of cytokeratin (green) and CC10 (red) (FIG. 8L). In regions of ECM there were areas of complex tissue formation after 4 weeks of culture which showed expression of (red) cytokeratin and collagen I (green) (FIG. 8N), cytokeratin (green) (FIG. 8O), CD31 (red) and lamnin (green) and DAPI nuclear stain in all (blue) (FIG. 8P). Flow cytometry contour plots of fetal lung cells (FIGS. 8Q-8S). Isotype control (FIG. 8Q) stained for expression of CD140a and Oct-4 (FIG. 8R) or CD140a and SSEA-4 (FIG. 8S). Confocal image of 7 um frozen sections of rat DC lung seeded with human fetal lung cells which were cultured for 2 weeks and then stained with DAPI (blue) to show presence of nuclei (FIG. 8T). Expression of CC10 (green) (FIG. 8U) and CD140a (red) and DAPI nuclear stain (blue) (FIG. 8V). Magnification 200× (FIG. 8B), 400× (FIGS. 8A, 8C-8H, 8J-8L, and 8N-8V) and 630× (FIGS. 8I and 8M).

FIGS. 9A-9R are drawn to recellularized rat lung after 21 days of culture (FIG. 9A) Gross image of recellularized rat lung after 21 days of culture. Region 1 includes the trachea, region 2 corresponds to the carina and upper bronchi, region 3 includes both bronchi and bronchioles, and region 4 is distal lung. The following micrographs correspond to the general regions outlined in this FIG. 1-4). (FIG. 9B) Phase contrast image of differentiated mESC in the trachea (in region 1) showing sheets of cells lining the trachea. Magnification 100×. Confocal images of 7 um frozen sections of regions 1 and 2 of recellularized lung matrix after 21 days of bioreactor culture demonstrating mESC differentiation. (FIG. 9C) Control for FIG. D (bar 20 um) stained with secondary antibody rabbit anti-mouse IgG conjugated to Alexa Fluor 488 and (FIG. 9D) cytokeratin-18 in cells lining the trachea (bar 20 um). (FIG. 9E) Control for FIG. F (bar 20 um) stained with secondary antibody rabbit anti-mouse IgG conjugated to Alexa Fluor 488 followed by donkey anti-goat IgG conjugated to Alexa Fluor 680 and (FIG. 9F) expression of cytokeratin-18 (green) and CC10 (red) (bar 20 um). White arrow points to Clara cells with characteristic intracellular granular staining for CC10. Magnifications 630× (bar 20 um). Confocal images of predominant cell type found in region 2 are shown in (FIGS. 9G-9K) (FIG. 9G) Control for FIG. H stained with secondary goat anti rat-antibody conjugated to Alexa Fluor 680 and (FIG. 9H) expression of alpha-actin (red) found in small pockets near bronchi. Magnifications 400× (bar 20 um). (FIG. 9I) Control for FIG. J-L (bar 20 um) stained with rabbit anti mouse IgG conjugated to Alexa Fluor 488 secondary antibody (green), goat anti rabbit highly cross absorbed antibody conjugated to Alexa Fluor 555 (purple) and donkey anti goat conjugated to Alexa Fluor 680 (red) and (FIG. 9J) expression of cytokeratin-18 (green) by small isolated regions of ciliated tracheal epithelial cells lining area just above the carina (bar 20 um). Magnifications 400×. (FIG. 9K) Enlargement of area in white box to show (J) a small region of ciliated epithelia cells (see white arrow) (bar 20 um). (FIGS. 9L-9M) Confocal evaluation of sections from region 3 (bar 20 um). (FIG. 9L) Formation of stratified tissue showing production of TTF-1 (purple) in cells lining the bronchial lumen (white arrow) along side of cells expressing CC10 (red) and cytokeratin-18 (green) (bar 20 um). Magnifications 400×. (M) Pan-cytokeratin (green) and CD31 (red) expression by cells found in region 3. White arrow points to line of CD31 positive endothelial cells which developed along side of the cytokeratin positive cells. Magnification 200×. (N-Q) Confocal evaluation of sections from region 4. (N) Isotype control for IgG APC and (O) expression of CD31 (red) by developing endothelial cells. Magnifications 630×. (P) Control stained with Rhodamine red X donkey anti-rabbit IgG and (O) expression of proSPC (red) positive cells in cyst like structures (bar 20 um). Magnifications 630×. (R) Immunoprecipation of surfactant protein A by cells isolated from normal lung, AC rat lung, or a 21 day culture of mESC recellularized lung. (D-R) Tissues were counterstained with DAPI to view nuclei (blue). Abbreviations: Clara cell protein 10, CC10; thyroid transcription factor-1, TTF-1; molecular weight (MW), normal lung (NL), decellularized lung (DL) and recellularized lung (RL).

### DETAILED DESCRIPTION OF THE INVENTION

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof.

As used herein, the term “subject” refers to any recipient of the compositions or implantable compositions described herein.

In one embodiment of the present invention there is provided a process for producing decellularized lung extracellular matrix (DC lung), comprising inducing cellular damage to native lung tissue; and removing cellular debris produced by the cellular damage to the lung tissue, wherein the remaining tissue is a decellularized lung extracellular matrix.

In a further embodiment the process comprises seeding onto or into the DC lung ECM one or both of endogenous progenitor lung cells to produce a cell:matrix construct. In another further embodiment the process comprises culturing the cell:matrix construct in vitro in a bioreactor under conditions effective to induce differentiation and growth of the cells toward a lung lineage within the matrix thereby producing functional lung tissue. In yet another further embodiment the process comprises implanting the functional lung tissue at one or more non-functioning sites of interest within a lung to restore at least some function thereto. In preferred embodiments the native lung tissue may comprise mammalian trachea and lungs.

In one aspect of these embodiments the step of inducing cellular damage comprises alternating cycles of rapid freezing and rapid thawing of native lung tissue or sonicating the native lung tissue. In another aspect the step of removing cellular debris comprises contacting the damaged lung tissue with a detergent or with peracetic acid within a continuously rotating bioreactor. In this aspect the detergent is 1% SDS continually circulated within the rotating bioreactor for about 5 weeks. Further to this aspect the step of removing cellular debris comprises treating any remaining damaged or intact cells with DNAase and RNAase.

In another embodiment of the present invention there is provided a method for producing engineered functional three-dimensional lung tissue, comprising decellularizing native lung tissue to produce a decellularized lung extracellular matrix (DC lung); isolating endogenous progenitor lung cells; seeding onto or into the DC lung the endogenous progenitor lung cells to produce a cell:matrix construct; and culturing the cell:matrix construct in a bioreactor under conditions effective to induce differentiation and growth of the cells toward a lung lineage within the matrix thereby producing the engineered functional three-dimensional lung tissue. Further to this embodiment the method comprises implanting the engineered lung tissue into a subject having a pulmonary disease, a pulmonary disorder or an injury to pulmonary tissue.

In both embodiments the engineered lung tissue may comprise cell types and cell numbers corresponding to native lung tissue. Representative cell types are type 1 epithelial cells, type 2 epithelial cells or endothelial cells.

In yet another embodiment of the present invention there is provided a composition comprising decellularized lung extracellular matrix and endogenous lung progenitor cells seeded thereon or therein. In this embodiment the mammalian lung may be a human lung. In yet another embodiment of the present invention there is provided an implantable composition comprising decellularized lung extracellular matrix and engineered functional lung tissue differentiated from endogenous lung progenitor cells grown in or on the decellularized extracellular matrix. In this embodiment the endogenous lung progenitor cells may be human lung progenitor cells. In yet another embodiment of the present invention there is provided a method for treating a lung to restore function thereto in a subject in need of such treatment, comprising implanting into the lung of the subject the implantable composition described supra, wherein growth of the lung tissue comprising the implantable composition restores at least partial function to the lung. In this embodiment the subject may have a pulmonary disease, a pulmonary disorder or an injury or damage to pulmonary tissue.

Provided herein are processes to decellularize native lung tissue to produce a lung decellularized extracellular matrix (DC lung). The DC lung is effective to promote stem cell attachment, survival and differentiation compared to other natural and synthetic matrices. Thus, also provided are methods to produce engineered functional three-dimensional complex lung tissue equivalents within the DC lung using bioreactor culture. This is the first showing that a decellularized matrix is superior to synthetic matrices for better repopulation of the matrix and for maintaining the three dimensional orientation critical for extracellular matrix production and site specific differentiation.

Producing the DC lung requires a combination of physical, mechanical and enzymatic processes to cause cellular damage with subsequent removal of cellular debris. Particularly, the process utilizes rapid freeze/thaw cycles to damage cells comprising the native lung tissue, for example, trachea and lung tissue. Placing the cellularly damaged lung tissue into a continuously rotating bioreactor allows a detergent or an agent such as peracetic acid to continuously circulate and contact the damaged tissue to effect removal of cells, damaged cells, including nuclei and nuclear material, and other cellular debris. Enzymes DNAase and RNAase effect removal of any remaining nuclear material.

The present invention also provides methods for producing an engineered functional three-dimensional lung tissue using the DC lung matrix and bioreactor culture. The importance of the bioreactor technique lies in its simulation of the fetal environment seen by the lung stem cells during organogenesis as they are being immersed in amniotic fluid. It is therefore a reasonable assumption that the bioreactor culture of engineered tissues may be better suited to the production of 3D tissues than would standard 2D plate culture since development of the tissue constructs in a liquid environment simulates amniotic fluid in that it accomplishes numerous functions for the developing tissues, such as: 1) maintaining a relatively constant temperature for the environment surrounding the cell/matrix constructs 2) permitting the easy delivery of growth factors to promote proper lung development and 3) ability to eliminate any cellular bio-products thereby minimizing cellular stress.

Bioreactors have been developed in various designs and capacities for different biotechnological applications yet their application in lung tissue growth or lung tissue culturing has not been attempted. Thus, the present invention provides the first use of a decellularized native lung for the in vitro differentiation of murine embryonic stem cells (mESC), human fetal lung stem cells (hFLSC) and murine/human somatic or endogenous lung progenitor cells (ELPS's) towards lung lineages and the first demonstration that DC lung matrices may be repopulated in a bioreactor. Bioreactors are commercially available and well-known in the art.

Particularly, endogenous progenitor lung cells or other stem cells obtained from a mammalian source. These progenitor or stem cells are seeded into or onto the DC lung matrix to form a cell:matrix construct. Culturing the cell:matrix construct in a rotating bioreactor induces attachment, differentiation and growth of the endogenous lung progenitor cells or stem cells into lung lineages. As such the present invention provides compositions or implantable compositions produced using the processes and methods described herein. One composition comprises the decellularized lung extracellular matrix with the endogenous lung progenitor cells seeded therein or thereon. A related composition is an implantable composition comprising the decellularized lung extracellular matrix and engineered functional lung tissue differentiated from endogenous lung progenitor cells grown therein or thereon.

In addition, the present invention provides methods of treating a lung to restore at least partial function thereto in a subject who has a pulmonary disease, a pulmonary disorder or damage or injury to pulmonary tissue. The implantable composition may be implanted into one or more non-functional sites of interest within the subject's lung to restore some, if not all, function to the affected area. One of ordinary skill in the art is well able to determine how much engineered functional lung tissue is to be implanted and which sites should receive one or more transplants. It is well known that such clinical protocols would depend on the subjects age, sex, the type and extent of pulmonary disease or disorder or damage or injuries incurred by the subject, the overall health of the subject and any drug regimens or treatments prescribed for the subject.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### Example 1

### Tissue Decellularization Procedure

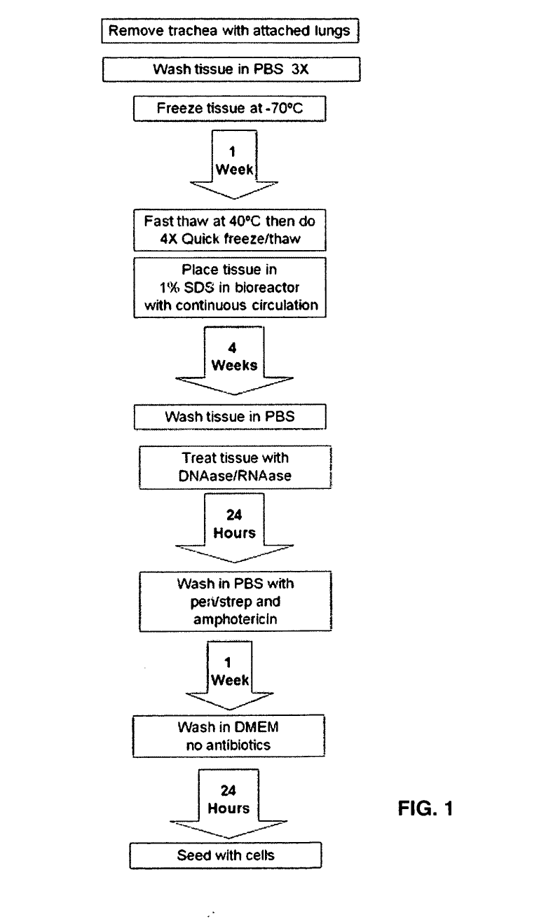

To develop the decellularization procedure, 96 sets of rat trachea with attached lungs were excised and cell membranes and nuclear material were removed using a process which combined freezing, enzymatic digestion and detergent treatment (FIG. 1). Whole trachea, esophagus and lungs were excised and tissues were cleaned to remove attached esophageal, lymphatic and connective tissues before lungs were weighed and photographed. Lungs were stored at −70° C. until decellularization was initiated. Lungs were later thawed in a 40° C. water bath and were flash frozen on dry ice followed by quick thawing, a process which was repeated four times, to enhance cell damage and facilitate cell loss. 3 mls of 2% sodium dodecyl sulfate (SDS) (Sigma, St. Louis, Mo.) was then injected into the trachea and into the right and left bronchus prior to placing the lungs into a 50 ml chamber of a rotating bioreactor (Synthecon, Houston, Tex.) containing 1% SDS for 1, 2, 3, 4 or 5 weeks at room temperature. Tissues were maintained at a rotational speed of 2.5 rpm which provided constant mechanical agitation of the tissue and chambers were allowed continuous circulation of fresh 1% SDS and removal of cell debris. Confocal microscopy was used to evaluate loss of cell membrane-associated major histocompatibility molecules-1 (MHC) and nucleic acids. Verification of cell removal was done by staining for residual membrane expressing MHC-1 as well as for presence of DNA using 4′,6-diamidino-2-phenylindole (DAPI) (Molecular Probes, Eugene, Oreg.) or propidium iodide (PI) staining (Sigma, St. Louis, Mo.). In the examination of loss of MHC-1 and DAPI staining each slide was stained with DAPI and human or rat specific MHC-1 and slides were then scored by counting positively DAPI stained nuclei or MHC-1 positive areas in five high-powered fields (hpfs) under ×400 magnification. Results were confirmed through a second reading by another person. At least 3 replicate measurements of each slide was performed by the same observer and 10 randomly selected slides were chosen from each set of serial sections through the entire piece of lung. Images were selected for inclusion in the manuscript based on scoring of the slides.

After five weeks of SDS treatment tissues were washed and placed in a mixture of 0.02 mg/ml DNAase and 20 ug/ml RNAase (Sigma, St. Louis, Mo.) in the bioreactor chamber for 24 hours at 37° C. Then whole trachea and lungs were washed for 24 hours in Dulbeco's phosphate buffered saline solution (DPBS) with antibiotics (streptomycin [90 ug/ml], penicillin [50 U/ml], and the antimycotic amphotericin B [25 ug/ml]; (Gibco Industries, Langley, Okla.) in a 50 ml rotary bioreactor chamber at room temperature. Finally DC lungs were refrigerated at 4° C. for one week or frozen at −70 C until used. Prior to recellularization with mESC DC lungs were washed with DMEM-F12 for 24 hours in a rotary bioreactor. For matrix comparison studies portions of distal lung were cut into 0.5 cm pieces prior to recellularization as described below. For these experiments rat lung was used instead of mouse lung due to the larger size of the rat tissues which made the processes of excision, decellularization and recellularization easier to accomplish. Use of rat lungs also allowed us to produce large pieces of distal lung for studies to compare DC lung with other matrix materials.

### Murine Embryonic Stem Cells

C57BL6 (F)+129/vs. mESC were purchased from Open Biosystems (Huntsville, Ala.). Post-cryogenic viability was determined to be approximately 80%. The embryonic feeder cells that were used were mitotically inactivated by treatment with mitomycin C by Open Biosystems prior to shipment. MESCs (Open Biosystems) were cultured on appropriate feeder cells as previously described by the manufacturer. mESCs were maintained in a 6-well tissue culture treated plate on which mouse fibroblast feeder cells had been established. Wells were seeded at between 1.5×105 and 4×105 cells/well and split when confluency reached about 80-85%. Media was replenished or replaced daily. Cells were placed in DMEM with 15% FCS, and supplemented with L-glutamine, sodium pyruvate, nonessential amino acids, penicillin-streptomycin, b-mercaptoethanol and Leukemia Inhibitor Factor according to the manufacturer's recommendations. Cells were cultured at 37° C. with 5% CO2. Differentiation of embryoid bodies (EB) was accomplished by limited trypsin digestion of mESC colonies 24 hours post passage and suspension of the cells in non-tissue culture treated petri dishes (Corning-Costar, Corning, N.Y.). EBs were cultured in suspension for 10 days and then placed into gelatinized 6-well plates. EBs were grown for 15-25 days as necessary. Percent viability of mESCs collected from the embryoid bodies at time of use was 95%+ for all cultures. All embryonic and feeder cell lines were tested and shown to be mycoplasma negative before use.

### Seeding and Culture of Matrices

To evaluate the ability of DC rat lung to support attachment and survival of cells we compared growth of mESCs on DC lung to growth on Matrigel and Gelfoam and a type-I collagen Pluronic F-127 (PF-127) hydrogel matrix (collagen-I/PF-127) produced in our laboratory. Gelling of microfluidically patterned collagen-I has shown previously to produce 3D matrices that mimic natural lung ECM.18 Based on these studies we produced a collagen-I/PF-127 hydrogel matrix formed by mixing equal volumes of 8 mg/ml collagen-I (Sigma, St. Louis, Mo.) with 40% PF-127 and then pipetting the mixture into 0.5 cm3 plastic molds. The final matrices of 4 mg/ml collagen-I (Sigma, St. Louis, Mo.) with 20% PF-127 were allowed to solidify at room temperature prior to use.

Distal DC lung, Matrigel and Gelfoam were cut into six equal sized 0.5 cm3 pieces. 2×106 mESC suspended in 0.1 ml of PF-127 hydrogel were injected through a 20 gage catheter into the center of each of the six 0.5 cm3 pieces of DC lung, Matrigel, Gelfoam or into six 0.5 cm3 pieces of collagen-I/PF-127 matrix. The seeding process was followed by a 5 minute centrifugation at 800 rpm (72 RCF) to help spread cells throughout the matrix. The mESC/DC matrix constructs were then placed into a 24 well culture dish that contained cell culture medium (see below) to allow for binding of mESC to the matrix materials before bioreactor culture. Constructs were cultured at 37° C. in a 5% CO2 incubator for 24 hours before placing each group of six cell/matrix constructs in a separate rotary bioreactor chamber containing lung differentiation medium for 6 days. For 2D plate culture of mESCs 2×106 cells per well were cultured in cell culture medium (above) or in lung differentiation medium in 24-well plates.

Seeding and Growth of Cells on Whole DC lung

For recellularization of whole trachea with attached lungs, 1×106 mESC suspended in 0.5 ml of PF-127 (15% in DMEM) were injected into the right and left main bronchus of the DC lung using a 20 gauge catheter for a total of 2×106 cells/lung. The seeding process was followed by a 5 minute centrifugation at 800 rpm (72 RCF) to help spread cells throughout the lung. The lungs were then placed into a 50 ml rotating bioreactor (Synthecon, Houston, Tex.) chamber that contained lung differentiation medium and were cultured at 37° C. in a CO2 incubator for 14 or 21 days. Media was circulated by a continuous pump action of the bioreactor at a speed of 2.0 rpm. Five lungs were harvested after 14 days of culture and the remaining 5 lungs were harvested after 21 days. Lungs were fixed in 4% paraformaldehyde (PAF) and evaluated as described below.

### Lung Cell Culture Medium and Lung Differentiation Medium

Lung cell culture medium was made as previously described 3 using DMEM/F-12 (Mediatech INC. Manassas, Va.) with addition of 33 mM glucose (Sigma, St. Louis, Mo.), insulin [20 ug/ml] (Sigma, St. Louis, Mo.), transferin [10 ug/ml] (Sigma, St. Louis, Mo.), selenium [100 nM] (Sigma, St. Louis, Mo.), putrescine [10 mM] (Sigma, St. Louis, Mo.), epidermal growth factor [20 ng/ml] (EGF, PeproTech, Rocky Hill, N.J.) and fibroblast growth factor [20 ng/mL] (FGF, Collaborative Biomedical, Bedford, Mass.). Lung differentiation medium was made using DMEM/F-12 (Mediatech INC. Manassas, Va.) with addition of 33 mM glucose (Sigma, St. Louis, Mo.), insulin [20 mg/ml] (Sigma, St. Louis, Mo.), transferin [10 mg/ml] (Sigma, St. Louis, Mo.), selenium [100 nM] (Sigma, St. Louis, Mo.), putrescine [10 mM](Sigma, St. Louis, Mo.), epidermal growth factor [25 ng/ml] (EGF, PeproTech, Rocky Hill, N.J.), insulin-like growth factor-1 [5 ng/ml](IGF, PeproTech, Rocky Hill, N.J.), Activin A [30 ng/ml] (SBH Biosciences, Natick, Mass. or R&D Biosystems, Minneapolis, Minn.) and lung homogenate [40_l/ml]. Lung homogenate was made by homogenizing 10 sets of C57B6 mouse lungs in 5 mls of lung differentiation medium. Homogenate was centrifuged to remove large pieces of cell debris and the resulting supernatant was filtered through a 4 um and then through a 1 um filter. Lung homogenate was stored at −70° C. until used. Lung differentiation media containing Activin A and lung homogenate were constantly circulated through the bioreactor chamber. Two days after initiation of bioreactor culture use of Activin A was discontinued and FGF 2, 7 and 10 [25 ng/ml each] (Collaborative Biomedical, Bedford, Mass.) were added to the circulating medium for the remainder of the culture period (14 or 21 days).

### Quantitation of DNA in DC Rat Lung

DAPI or PI staining was used to document the loss of nuclei or DNA in lung tissue during the process of decellularization. For staining with DAPI, a DAPI stock solution [5 mg/ml] (14.3 mM for the dihydrochloride or 10.9 mM for the dilactate), was made by dissolving the contents of one vial (10 mg of DAPI) in 2 ml of deionized water (dH2O) which was followed by sonication for 2 hours. For long-term storage the aliquots were stored at −20° C. For PI staining PI stock was made by dissolving PI [1 mg] in 1 ml dH2O which was stored at 4° C. Working PI stain was made by adding PI [200 μl of 1 mg/ml] (Sigma, BioSure, Molecular Probes) to 10 ml of Triton X-100 [0.1% (v/v)] (Sigma, St. Louis, Mo.) in PBS. Sections were incubated with dilute stain, for 5 minutes, rinsed several times in PBS and then drained and mounted in Molecular Probes' SlowFade® Antifade Kit, SlowFade Light Antifade Kit or ProLong® Antifade Kit.

To determine if DNA remained in the fully acellularized rat lung strips of matrix were treated as described (19). Strips of DC lung were digested with proteinase K, extracted with phenol-chloroform-isoamyl alcohol (25:24:1) and aqueous layers were removed and ethanol precipitated at −20° C. for 12 hours to isolate any DNA present. The remainder of the protocol was followed as described and samples were separated by electrophoresis on a 3% LMP agarose gel with ethidium bromide at 60V for one hour, and visualized with ultraviolet transillumination.

Evaluation of mESC/Matrix Constructs

Three-dimensional (3D) mESC/DC construct culture in a bioreactor was compared to 2D plate culture of mESC with and without the addition of lung differentiation medium. Six 0.5 cm3 pieces of DC lung, Matrigel, Gelfoam or collagen-I/PF-127 mESC/constructs were cultured in separate bioreactor chambers and were harvested after 7 days of culture. One quarter of each matrix was removed and sectioned to allow for microscopic examination of cell attachment to each matrix. Each portion of matrix was frozen in tissue freezing medium (Triangle Biomedical Sciences, Durham, N.C.) and serially sectioned on a Microm cryomicrotome (Thermo Scientific, Walldorf, Germany). To determine the distribution of cells, each slide was stained with DAPI and was then scored by counting positively DAPI stained nuclei in five high-powered fields (hpfs) under ×400 magnification. All slides were counted without knowledge of the matrix being examined, and results were confirmed through a second reading by another person. At least 3 replicate measurements of DAPI+ cells were performed by the same observer in 10 randomly selected slides.

The remaining cells from each individual mESC/matrix construct were collected after suspension of each matrix in 1 ml of 0.25% trypsin for 5 minutes followed by physical disruption by running each piece of construct against a fine mesh screen.

Cells from each piece of mESC/matrix construct were evaluated for cell viability, induction of apoptosis and lung specific differentiation after immunohistochemical staining with analysis by flow cytometry. Viability was analyzed by vital fluorescent staining (calcein-AM and ethidium homodimer-1; Molecular Probes Eugene, Oreg.). Apoptosis in samples was determined by quantitation of DNA strand breaks using the TUNEL Assay (In Situ Cell Death Kit, Boehringer Mannheim, Mannheim, Germany) as described in the manufacturer's instructions. The results from the evaluation of the influence of matrix on cell survival, apoptosis and differentiation were compared to averages of 2D culture of 6 wells containing 2×106 mESC per well cultured in 24-well tissue culture plates with the addition of lung differentiation medium. 2D and 3D cultured mESCs were also examined for lung epithelial or endothelial lineage selection by immunostaining for expression of cytokeratin 18, CD31 and pro-SPC with analysis using flow cytometry.

### Immunohistochemistry

For immunostaining, cell matrix constructs or whole lungs were fixed in 2% paraformaldehyde (PAF) in PBS overnight at room temperature. Portions of matrix constructs or lungs were frozen in tissue freezing medium (Triangle Biomedical Sciences, Durham, N.C.) and sectioned on a Microm cryomicrotome (Thermo Scientific, Walldorf, Germany). Evaluation of rat and murine major histocompatibility-1 (MHC-1), Human MHC-1, CD31 and CD140a were done as described by the manufacturer (BD Biosystems) using antibodies directly conjugated to fluoroscein isothyocyanate (FITC), allophycocyanin (APC) or perCP-cyanin-5 (PerCp-Cy-5). Primary antibodies and dilutions used were anti-collagen-I (goat, 1/250, Santa Cruz Biologicals, Santa Cruz, Calif.), anti-elastin (1/150, Santa Cruz Biologicals, Santa Cruz, Calif.), cytokeratin-18 (mouse 1:400, Chemicon, Temecula, Calif.), anti-collagen-IV (goat, 1/200 (1/250, Santa Cruz Biologicals, Santa Cruz, Calif.), pro-surfactant protein-C (pro-SPC) (rabbit, 1/200, Chemicon (Temecula, Calif.), Clara cell protein-10 (CC10) (goat, 1/250, Santa Cruz Biologicals, Santa Cruz, Calif.), anti-laminin (goat, 1/200, Santa Cruz Biologicals, Santa Cruz, Calif.), alpha-actin (rat monoclonal, 1/200, Abcam, Cambridge, Mass.), thyroid transcription factor-1 (TTF-1) (rabbit, 1/200, Millipore, Billerica, Mass.) and pan-cytokeratin (mouse, 1:200, Chemicon, Temecula, Calif.).

Tissue sections were incubated with primary antibody for one hour at 4° C. in a humid chamber, washed and then incubated with secondary antibody for 30 min according to manufacturer's instructions. Staining of internal proteins was done after fixation of cells in PAF and incubation in BD Perm2 permeabilization solution as described by the manufacturer (BD Biosystems). For negative controls corresponding immunoglobulin and species matched (IgG)-matched isotype control antibodies were used or the primary antibodies were omitted and sections were stained with secondary antibodies alone in order to set baseline values for analysis markers or as tissue staining controls. Donkey anti-rabbit IgG conjugated to Rhodamine Red-X, donkey anti-goat IgG antibody conjugated to Alexa Fluor 680 (1:250; Invitrogen), rabbit anti-mouse IgG conjugated to Alexa Fluor 488 (1:400, Invitrogen), goat anti-rat IgG conjugated to Alexa Fluor 680 (1:250 dilution; Invitrogen), goat anti-rabbit highly cross absorbed, IgG conjugated to Alexa Fluor 555 (1:250 dilution; Invitrogen) and donkey anti-goat IgG conjugated to Alexa Fluor 594 (1:250 dilution; Invitrogen) were used as secondary antibodies.

Fluorescent Microscopy, Confocal Microscopy and/or Flow Cytometry Analysis

Fluorescent microscopy was done using a Zeiss Axioscope Fluorescent microscope (Oberkochen, Germany) or a Nikon T300 Inverted Fluorescent microscope (Nikon Corp., Melville, N.Y.). Confocal microscopy was done on a Zeiss LSM 510 UV-META Confocal microscope. For flow cytometry, cells were fixed with 2% PAF before analysis using a FACSAria instrument (BD Biosciences, San Jose Calif.), with acquisition and analysis using the FACSDiva program (BD Biosciences). For each sample 10,000-20,000 cells were collected.

### Two-Photon Microscopy

Two-photon microscopy was done with a Zeiss 410 Confocal Laser Scanning Microscope. Lung samples were imaged using multiphoton microscopy to detect tissue autofluorescence and second harmonic generation microscopy. Briefly, multiphoton excitation was from a titanium:sapphire laser (Tsunami, SpectraPhysics, Mountainview, Calif.) centered at 780 nm routed into the scanhead and through the sample objective. Fluorescence emission collected from the sample was detected in a nondescanned configuration using cooled PMTs (Hamamatsu, USA). Fluorescence emission in the spectral region of 500-650 nm was collected for detection of broadband autofluorescence from the lung. Second harmonic generation was collected using 800 nm excitation and a 400±14 nm bandpass filter in the nondescanned detector path. The lung was placed on an imaging dish having a #1.5 coverslip and immersed in phosphate buffered saline. Several sites at the apex of the lobe and the broncho-alveolar region were chosen. At each site z-stack was obtained from the outer lung surface using a z-interval of 1 um and 150 um total depths using a 40×, 0.75 N.A. water immersion objective which provided a field of view of 320×320 um.

### Immunoprecipitation of Surfactant Protein A

Cells isolated from normal mouse lung, DC rat lung or 21-day DC rat lung cultured with mESCs were washed with 10 ml of PBS in a conical tube and centrifuged at 400 xg for 10 minutes. The cell pellet was resuspended in 1 ml of cold RIPA lysis buffer [50 mM Tris HCl pH 8; 150 mM NaCl; 1% NP-40; 0.5% sodium deoxycholate] containing 1× Protease Inhibitor Cocktail (Sigma, St. Louis, Mo.). The lysate was centrifuged at 10,000 xg for 15 minutes at 4° C. The supernatant was collected and the cell lysate was frozen at this point for long-term storage at minus 70° C. Immunoprecipitation of surfactant protein A was performed following the protocol recommended by Abcam. In brief 50 μl of prepared Protein A (Millipore, Billerica, Mass.) slurry was added to 500 μl of cell lysate and was incubated on ice for 30-60 minutes to preclear the lysate. The sample was centrifuged at 10,000 xg for 10 minutes at 4° C. and supernatant transferred to a fresh eppendorf. 10 μg of antibody to surfactant protein A (Chemicon; Millipore, Billerica, Mass.) was added and the sample was incubated at 4° C. for 1 hour. Finally 50 μl of Protein A (Amersham, Pharmacia Biotech, Piscataway, N.J.) slurry was added and the sample was incubated for 1 hour at 4° C. on a rocking platform. The beads were collected and washed 3 times with 500 μl of Lysis Buffer. After the last wash, 50 μl of 1× Laemmli sample buffer was added to the bead pellet. The sample was vortexed and then heated to 90-100° C. for 10 minutes. The sample was centrifuged at 10,000 xg for 5 minutes, supernatant was collected and loaded onto an SDS PAGE gel. The gel was finally stained with comassi blue for visual analysis of the immunoprecipitated surfactant A protein.

### Statistical Analysis

Statistical analysis was performed using GraphPad InSTAT software (version 2003). Mean values and standard deviation are reported. Analysis of variance (ANOVA) was performed and data was subjected to Tukey Kramer multiple comparison test. Mean differences in the values were considered significant when p was less than 0.05.

### Example 2

### Process for Decellularization of Lung Tissue

Decellularization of lung tissue utilizes a combination of physical (freezing), mechanical (rotary bioreactor with re-circulating fluidics) and enzymatic (DNAase and RNAase treatment) steps to produce DC-lung that was free of cellular material, remnants of nuclei or nuclear material (FIG. 1). It is contemplated that sonication also may be utilized, and may be more effective, to remove cellular material and compare it to the freeze-thaw method described herein. Sonication may cause less damage to the ECM and allow for better retention of collagen. Also, it is contemplated that peracetic acid (PAA) may be used instead of a detergent-based, e.g., sodium dodecyl sulfate, in the decellularization process.

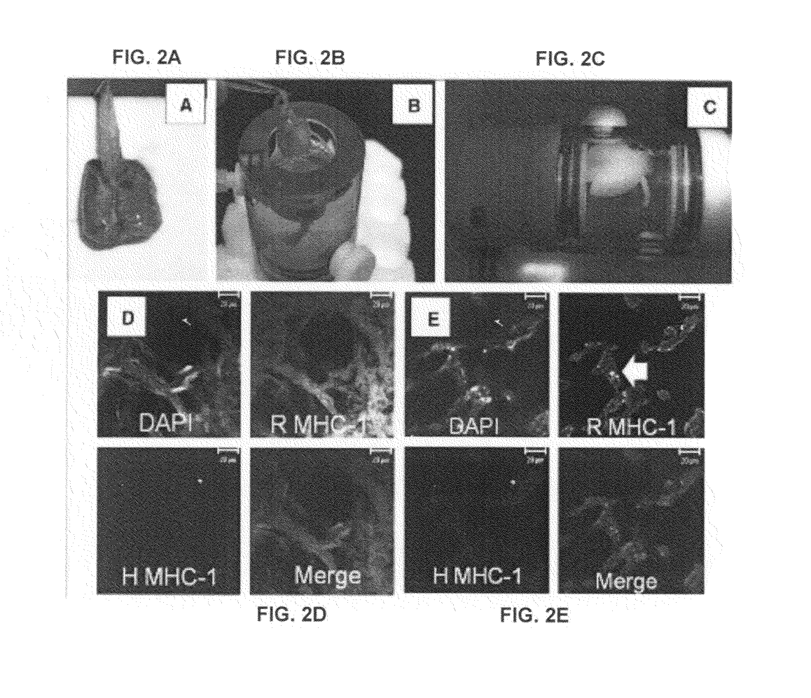

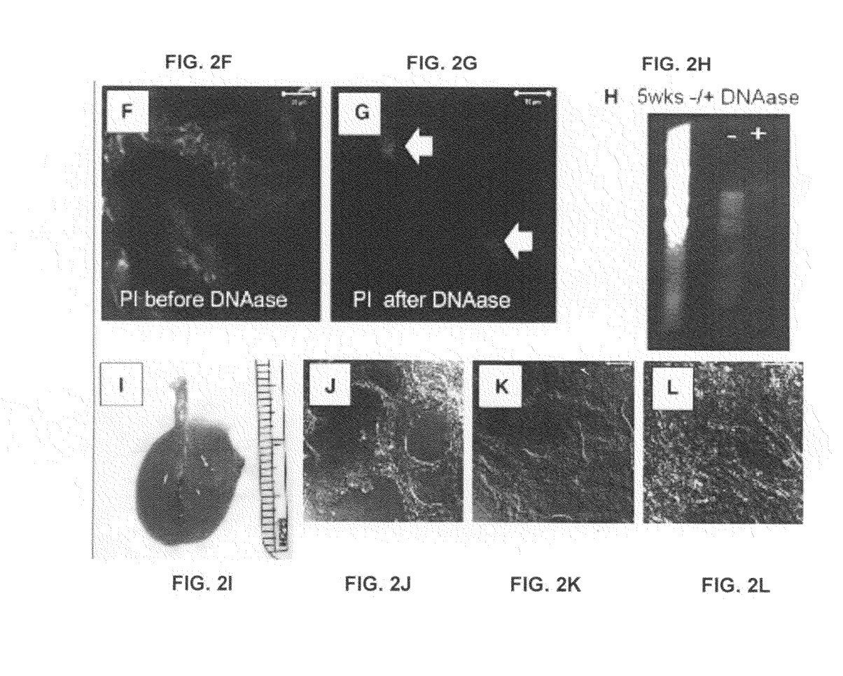

It was determined that lungs could be fully decellularized without destroying the underlying extracellular matrix (ECM). The trachea, esophagus and lungs were removed together and the excised tissues were later cleaned to remove attached esophageal and connective tissue. Tissues that were not frozen prior to detergent treatment or that were treated after freezing with polyethylene glycol or Triton X-100 retained the presence of nuclei and DNA after 4 weeks or showed signs of significant degradation of the lung ECM after detergent treatment (data not shown). Higher concentrations of SDS>2% resulted in significant loss of the natural ECM and influenced our final selection of 1% SDS for the detergent treatments. Whole rat lungs (FIG. 2A) were placed in a rotating bioreactor with continually circulated fresh 1% SDS in the chamber (FIGS. 2B and 2C). In order to reach regions deep within the organ 1% SDS was injected through the trachea three times a day to fully flush the tissues and increase removal of cell debris. Examination of the tissues after one week showed the presence of high levels of rat major histocompatibility complex (MHC-1) staining (FIG. 2D). DAPI staining of the tissues at this stage also showed that many intact nuclei were still present (FIG. 2D). After five weeks of SDS treatment there was no longer significant Rat MHC-1 staining (FIG. 2E) but DAPI or PI staining indicated that all of the nuclear material was not removed by the detergent treatments alone (FIGS. 2E and 2F). Because of this, tissues were treated with DNAase and RNAase for 24 hours which resulted in production of AC trachea and lung tissue that contained significantly less DNA as estimated by PI staining of tissue sections (FIG. 2G). Confocal analysis suggested that most DNA had been removed at this stage, and examination of the AC lung matrix by electrophoresis on 3% LMP agarose gels with ethidium bromide confirmed that only trace amounts of DNA remained in the AC rat lung (FIG. 2H) after decellularization using this protocol. The final AC lung tissue had a white glassy appearance (FIG. 2I). There was no positive staining for MHC-1 in the distal airway (FIG. 2J), middle airway (FIG. 2K), bronchus and trachea (FIG. 2L).

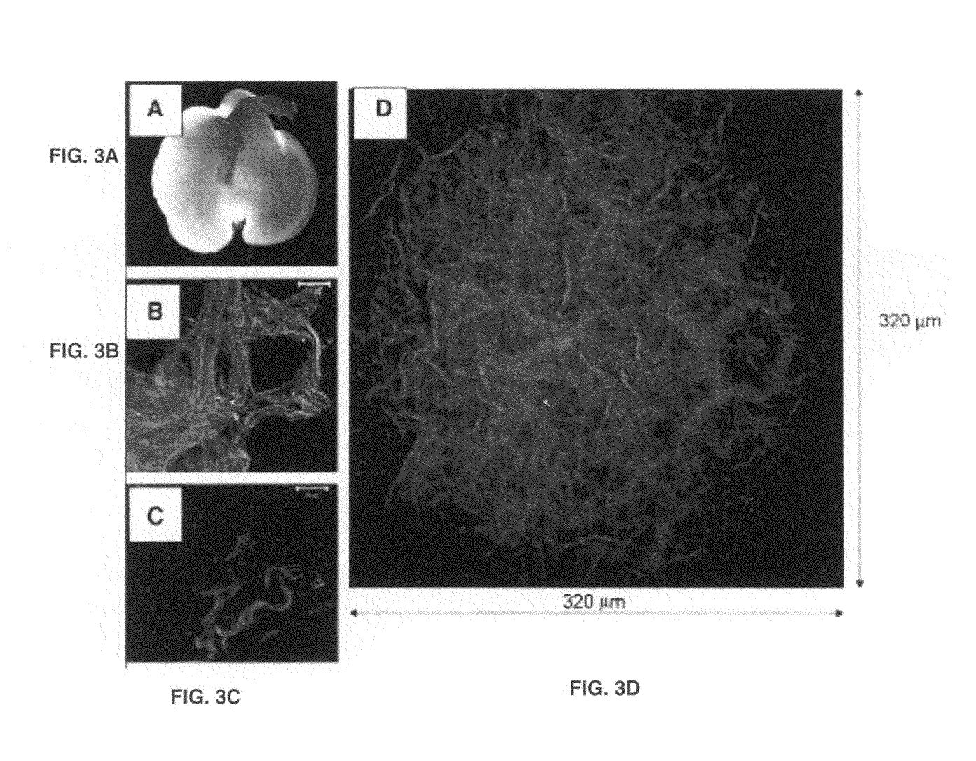

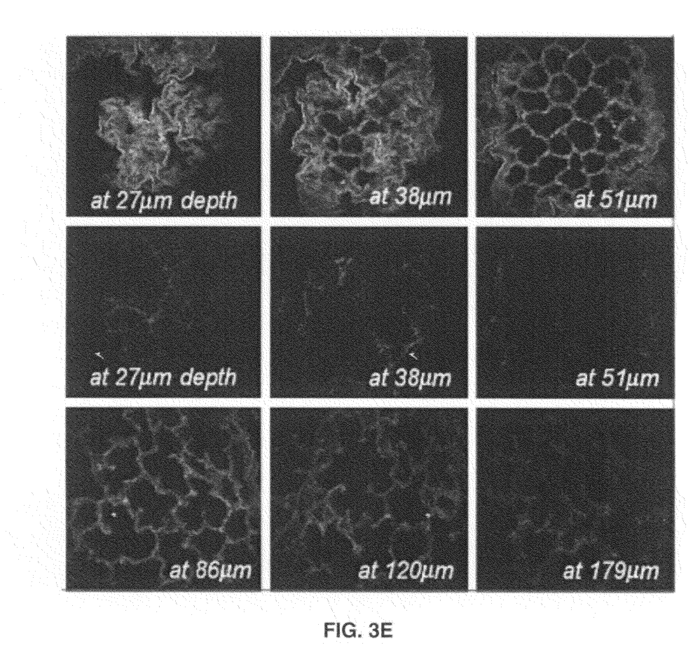

Prior to tissue culture AC rat lungs with attached trachea were washed in a combination of antibiotics and antimycotic for 24 hours, followed with a 24 hour wash in PBS and then a 24-hour wash in DMEM. After decellularization whole lungs still retained obvious signs of the trachea and branching bronchial ECM network within the tissues (FIG. 3A).

Collagen-I and elastin are the main components of the pulmonary interstitium and form the basis for the mechanical scaffold or matrix that maintains the integrity of the lung during the process of ventilation. Basement membrane also contains laminin and collagen-IV. In order to determine the influence of the decellularization on the AC lung ECM, 7 um frozen sections of AC lung were stained for expression of collagen-I, collagen-IV, elastin and laminin. Collagen-I (FIG. 3B green) and elastin (FIG. 3C, green) were found throughout the AC rat lung matrix although collagen-IV (FIG. 3B, red) and laminin (FIG. 3C, red) as well as fibronectin were lost in the decellularization process.

Two-photon microscopy was used to produce a 3D reconstruction of AC rat lung viewed from a 0 degree angle to a depth of 320 um. Autofluorescence (green) was combined with second harmonic generation (SHG-red) to show the relative makeup of the AC lung. SHG microscopy was used to visualize fibrilar collagen (red) in this 3D reconstructed z-projection (collapsed zstack) and cells and other ECM appear green (FIG. 3D). Two-photon microscopy at depths of 27, 38, 51, 86, 120 and 179 um in the AC lung demonstrated that the basic lung architecture was preserved as was the presence of type-I fibrilar collagen and that there were no intact cells present within the fully decellularized tissue (FIG. 3E).

### Example 3

### Evaluation of Stem Cell Attachment in DC Lung

To evaluate the ability of DC lung to support attachment and survival of cells DC lung was compared to matrices that have been shown to support development of lung tissue such as Matrigel and Gelfoam. A Collagen I/PF-127 hydrogel matrix which produced 3D fiber formations that were similar to what we saw in the DC lung (FIG. 1N-Q). The influence of composition and stiffness of matrices has been shown to be important to support of a number of biological processes as well as to significantly influence cell differentiation and tissue development.

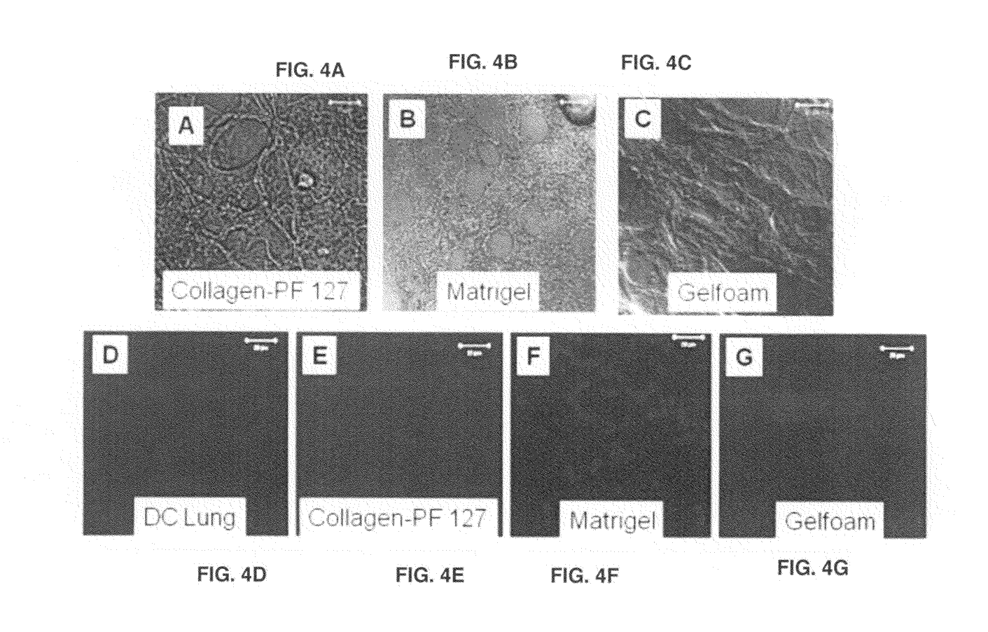

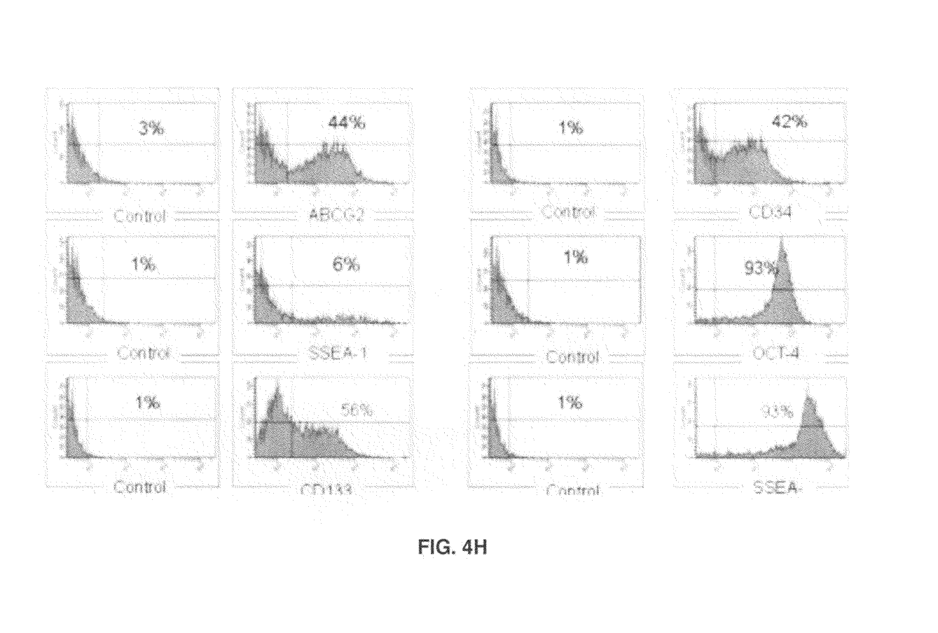

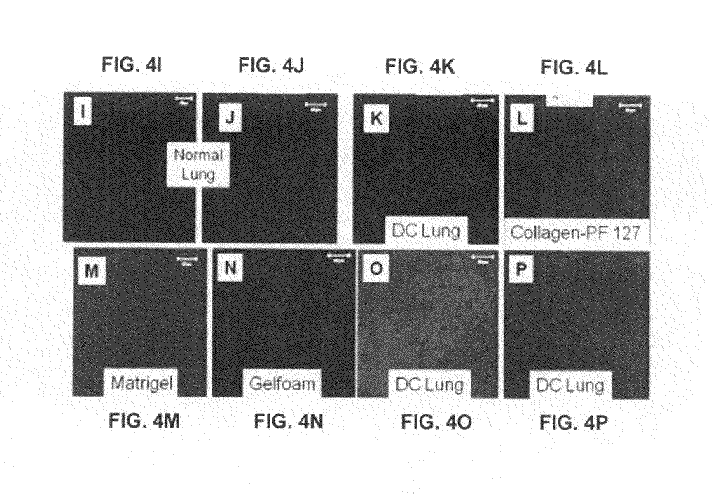

The ability of human endogenous stem cells, Oct-4+ SSEA-1+ murine embryonic stem cells and fetal lung cells to populate DC lung, collagen-1/PF127 hydrogel matrix, Matrigel and Gelfoam and to survive after 1 week of culture was examined. mESCs populated the DC lung matrix very well and cells were found throughout each 1 mm portion of matrix (FIG. 4D). Cells did not populate the other matrices as evenly and although the same number of cells was used to seed each matrix there were fewer cells overall found in any of the other matrices after 1 week of culture (FIG. 4E, collagen-PF127, FIG. 4B Matrigel and FIG. 4G Gelfoam).

Human endogenous lung progenitor cell (ELPC) are a heterogeneous population of lung specific stem cells which express Octogon-4 (Oct-4) and stage specific embryonic antigen-4 (SSEA-4) with variable expression of CD 133, CD34 and ABCG2 (FIG. 4H). It has been shown that this population of lung progenitor cells has the capacity to differentiate into cell types formed in normal lung when provided with appropriate growth factors and defined culture conditions. Attachment and survival of endogenous lung progenitor cells on DC lung, Gelfoam, Matrigel and collagen-I/PF-127 matrix were initially evaluated by DAPI staining and were then compared to normal lung.

As shown with mESCs (FIGS. 4D-4G) the DC lung matrix (FIG. 4K) supported attachment and survival of this population of lung derived ELPCs. The configuration and orientation of cells on DC lung matrix after 1 week of culture was similar to what is seen for native lung as was the deposition of cells in regions of trachea (FIG. 4O) and distal lung (FIG. 4P). The decellularization protocol completely removed all cell membranes, chondrocytes, MHC-class I expression, nuclei or nuclear material, but also was shown to retain some collagen I and elastin as well as a major portion of the ECM/lung substructure. Type II collagen staining for cartilage was negative in DC trachea and lung using this method. It is contemplated that a similar approach is useful to examine decellularization of lung tissue using other methods as mentioned above.

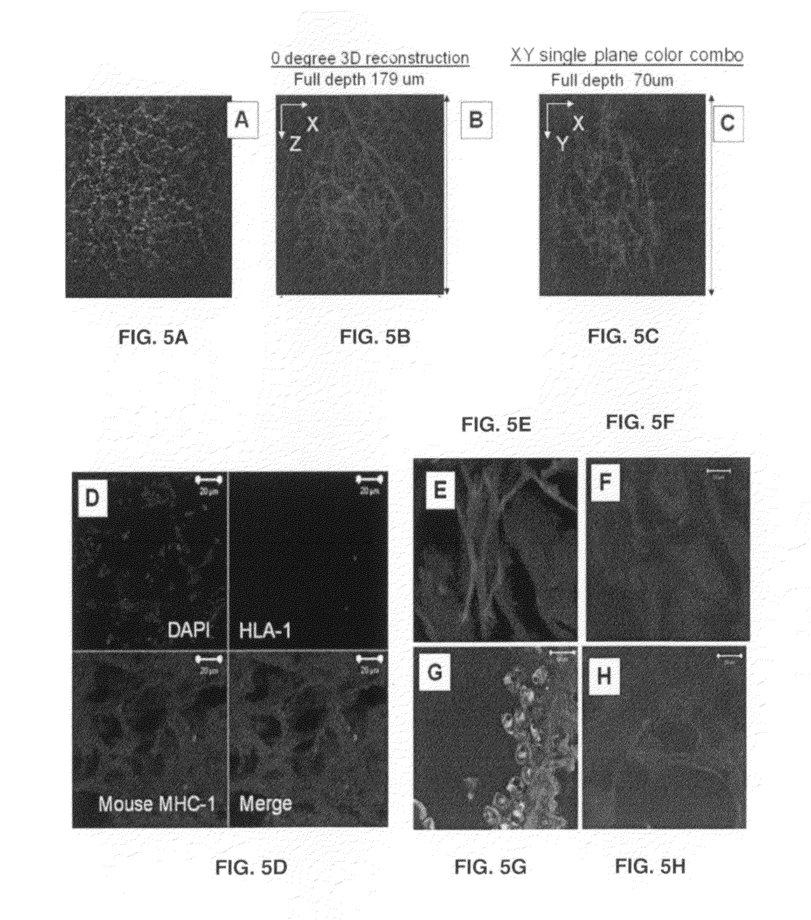

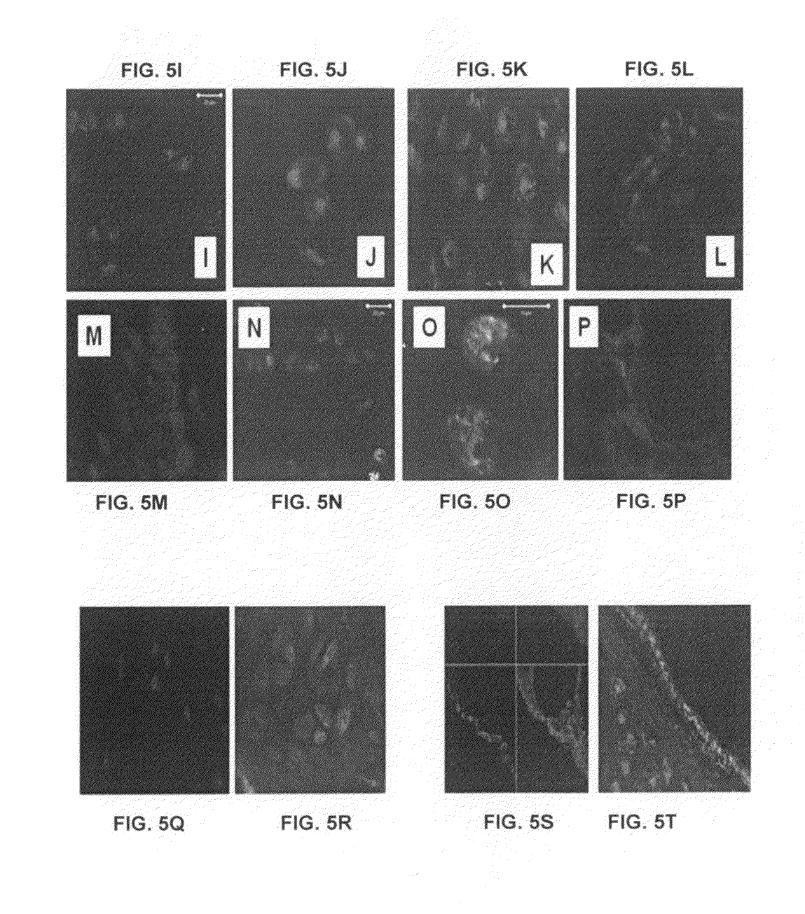

Decellularized biological tissues have been shown to provide for a more natural environment to support attachment, migration and tissue formation. 2 photon microscopic examination of normal lung (FIG. 5A) shows both the native form of the fibrilar collagen strands as well as the amount found in normal lung ECM (FIG. 5A). It was found that collagen did not maintain its wavy appearance and the majority of collagen remaining after removal of cells was predominantly part of the substructure of the bronchi/bronchioles. Elastin was found throughout the lung indicating that even after the process of decellularization, elastin fibers are plentiful in the decellularized matrix (FIG. 5B). This change in collagen might represent an effect on the collagen from the current protocol for the decellularization process itself. It is contemplated that peracetic acid treatment will allow for better retention of these components than our current SDS protocol. A similar approach may be used to examine ECM in peracetic acid treated lung tissue.

After seeding and culture of mESCs on the DC lung matrix expression of MHC-1 was observed again (FIG. 5D). There was significant contraction of the DC lung matrix after seeding of cells which occurred over a 2 day period, whereas no contraction was seen in any of the other matrices evaluated. mESCs differentiated into a variety of cell types after two weeks of culture. Cytokeratin positive epithelial cells (FIG. 5I), proSPC positive type II cells (FIG. 5J) and CC10 positive Clara cells (FIG. 5K) with characteristic staining of CC10 granules were found. Clumps of CD140a positive fibroblast cells were also present (FIG. 5L) in some parts of the DC lung construct. After three weeks in culture numerous mESCs were seen to express cytokeratin and there were also a few Aquaporin-5 positive cells suggesting that mESC were capable of differentiating into type I pneumocytes (FIG. 5N-5O) and expression of this or other mature lung cell markers is currently being validated by PCR, western blotting and immunoprecipitation.

In regions that had been trachea in the normal tissue extensive sheets of cytokeratin positive cells similar to what is found in normal lung were observed (FIG. 5R). There was also some localized expression of CD31, an endothelial cell marker (FIG. 5P) in the developing tissue with expression of cytokeratin in close proximity to the CD31+ endothelial cells reminiscent to what is seen in vivo during development of vascular tissue in the lung. In many of the recellularized constructs we saw evidence of complex tissue formation after longer periods in culture. FIG. 5T shows development of areas containing CC10+ cells in close proximity to regions of developing epithelium which were seen to express TTF-1a transcription factor (16) found in early stages of lung epithelial development.

### Example 4

### Comparison of mESC Growth on Selected Matrices

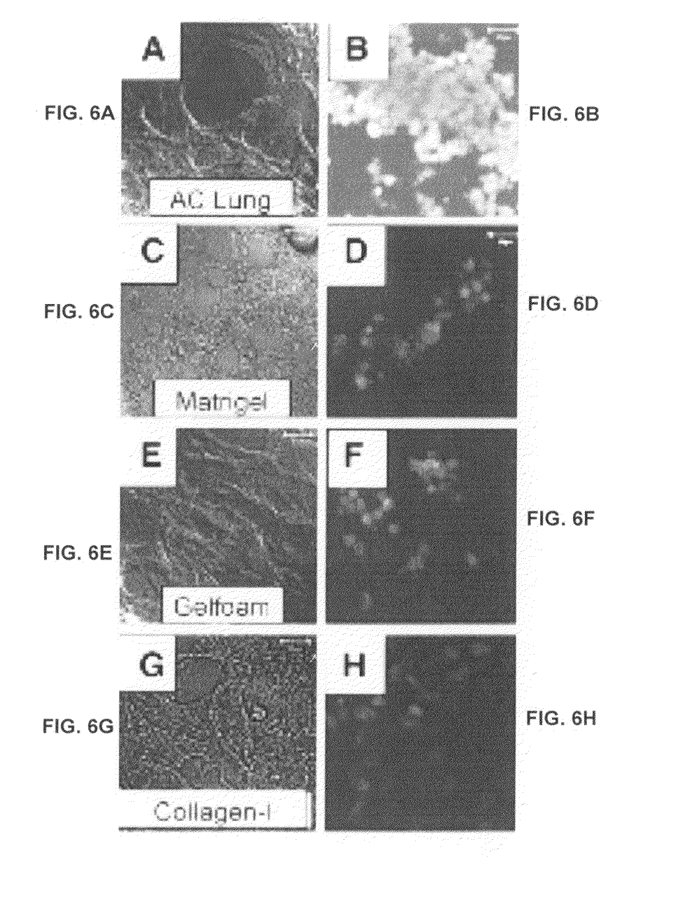

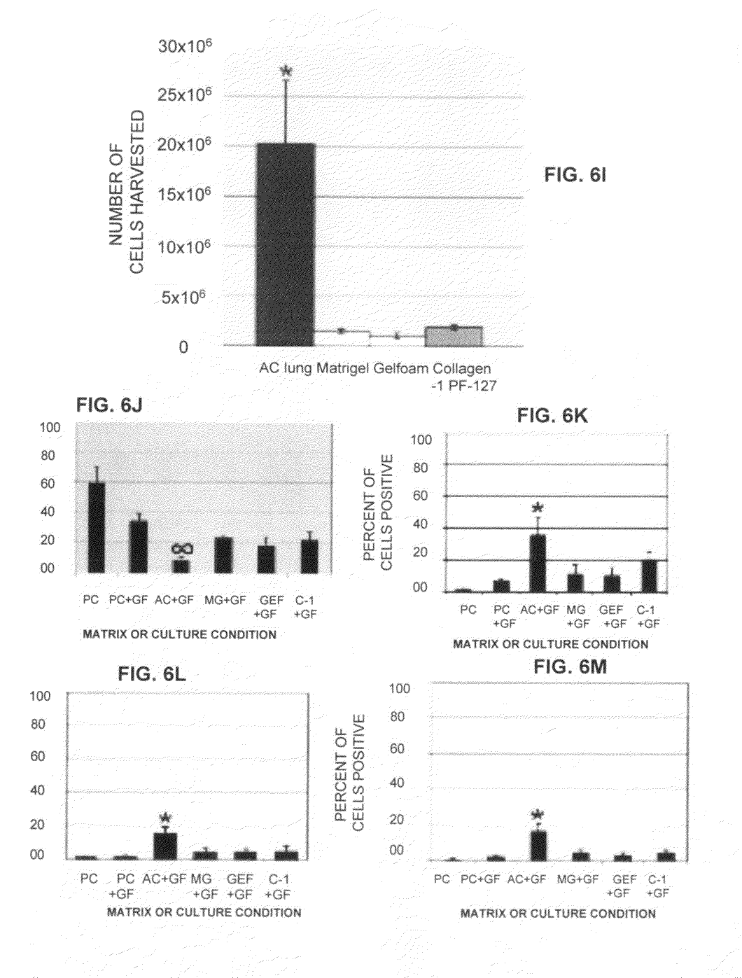

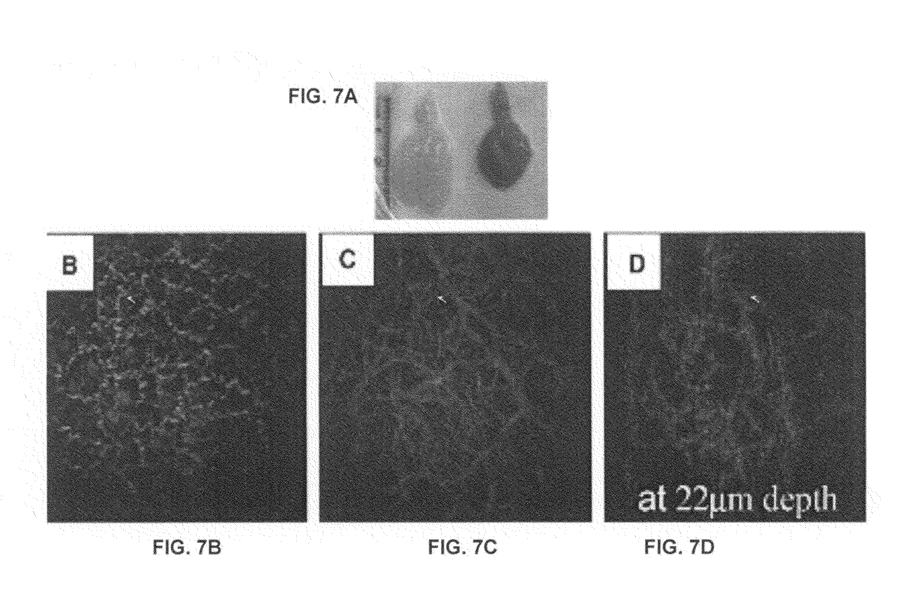

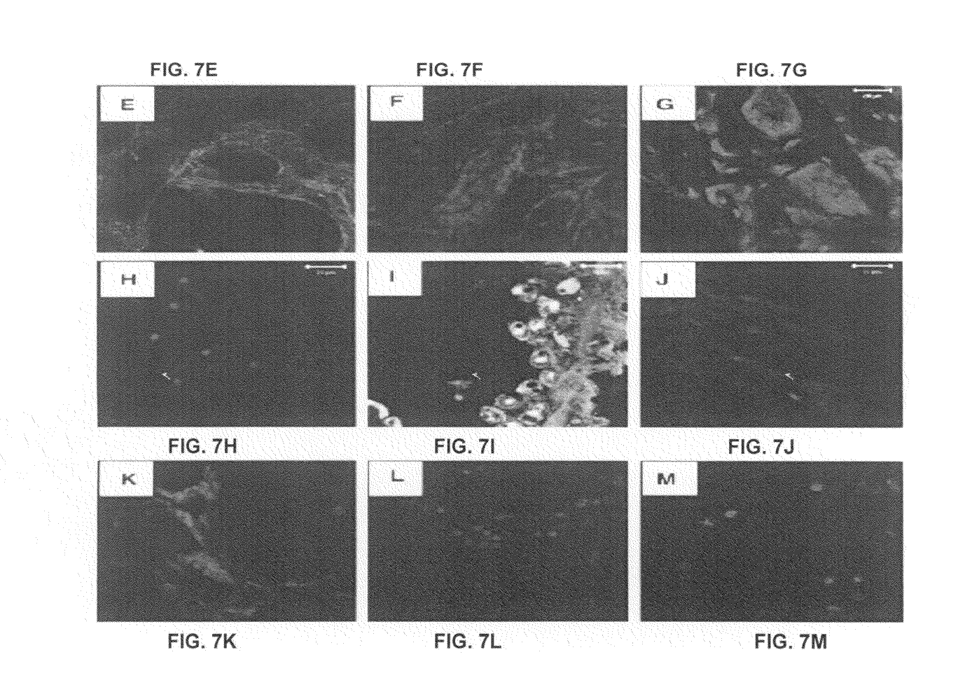

Sections of DC lung (FIG. 6A), Matrigel (FIG. 6C), Gelfoam (FIG. 6E) or the Type I collagen-UPF-127 hydrogel matrix (FIG. 6G) were examined using transmitted white light to show the substructure of each matrix material. This was done to evaluate the fibrilar nature and structural composition of each matrix. The influence of composition and rigidity of matrices has been shown to be important for support of a number of biological processes and to significantly influence cell differentiation and tissue development. Murine embryonic stem cells (mESC) were cultured on DC rat lung matrix (FIG. 6B) and compared cell attachment and survival of cells to mESC cultured on Matrigel (FIG. 6D), Gelfoam (FIG. 6F) or collagen-I/PF-127 hydrogel matrix (FIG. 6H). For measurements of DAPI+ stained tissue sections at least 3 replicate measurements of DAPI⁺ cells were performed by the same observer in 10 randomly selected slides which was followed by examination by a second observer. For these studies the intraobserver coefficient of variation was 6%, and the interobserver correlation coefficient was 0.8% for examination of these slides. Examination of DAPI stained slides showed that mESCs readily populated the DC lung matrix (FIG. 6B) and cells were found to be evenly spread throughout each 0.5 cm³ portion of this matrix. MESCs did not populate the other matrices evenly and fewer cells were retained by some materials although the same number of cells was used to seed each matrix (2×10⁶ mESCs). Flow cytometry results confirmed evaluations of slides which indicated that fewer cells were retained by the Matrigel (FIGS. 6D and 6I), Gelfoam (FIGS. 7F and 7I) or the collagen I/PF-127 matrix (FIGS. 6H and 6I) when compared to viable cell retention by DC lung (FIGS. 6B and 6I) (P<0.001). Cells cultured in 2D tissue culture plates with culture media or lung differentiation media had significantly higher levels of terminal deoxynucleotidyl transferase dUTP nick end labeling (TUNEL) positive (apoptotic) cells than cells cultured on any of the 3D matrices used (FIGS. 6I and 6J (P<0.001)). DC lung also had significantly fewer TUNEL positive (P<0.05) cells when compared to Matrigel and collagen-I/PF-127 but not when compared to Gelfoam (FIG. 6J).

Evaluation of mESCs cultured on DC lung showed significantly higher levels of cells differentiating towards the selected lung lineages which included expression of cytokeratin-18 (FIG. 6K) (P<0.001) by epithelial cells, CD31 (FIG. 6L) (P<0.001) by endothelial cells, and pro-SPC (FIG. 6M) (P<0.001) by type II pneumocytes.

### Example 5

### Examination of Whole Lung Repopulated with mESCs

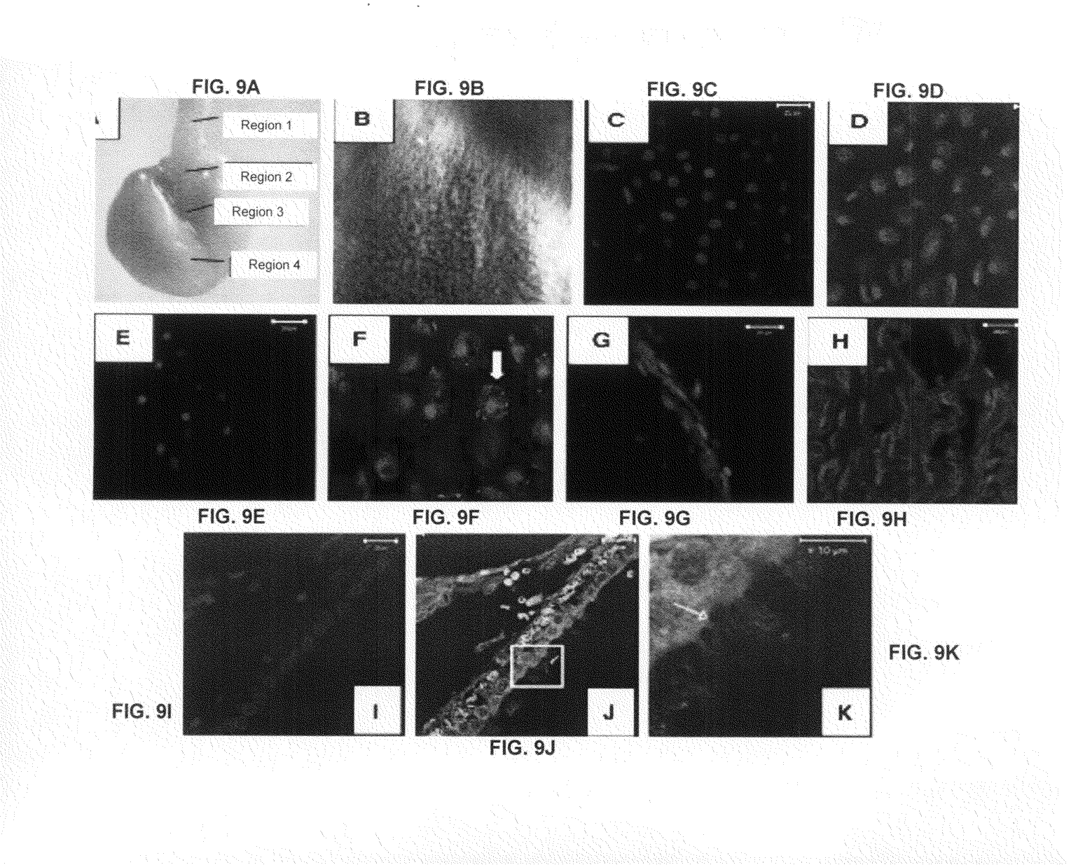

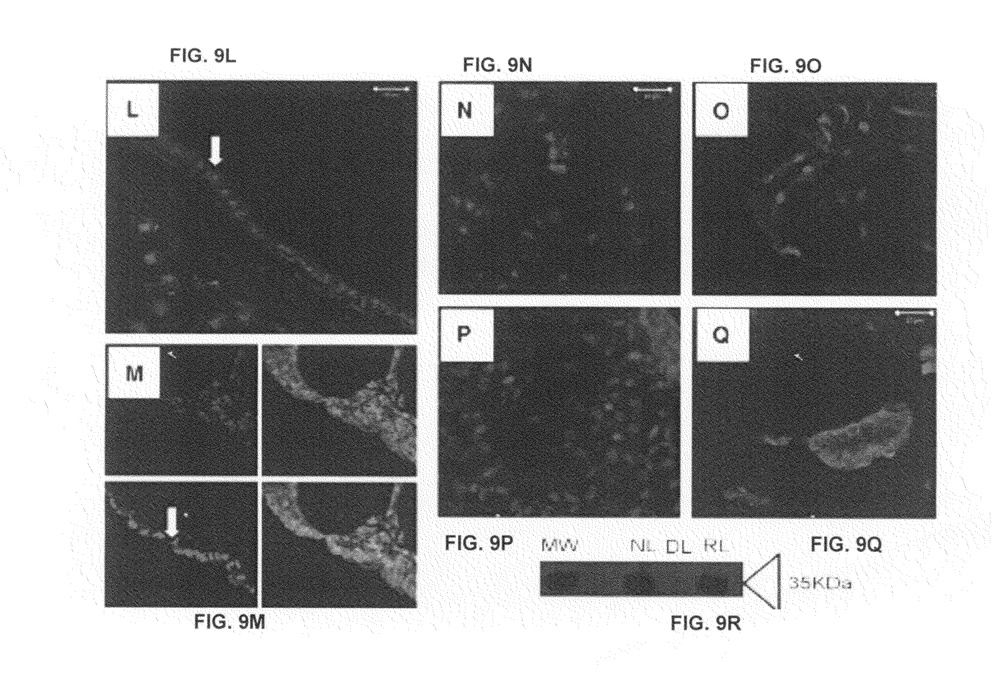

DC lungs (FIG. 7A, left) were seeded with murine embryonic stem cells (mESCs) and cultured for 14 (FIG. 7A) or 21 days (FIG. 9). Recellularization of whole trachea and lungs always resulted in considerable shrinkage of the DC tissues and the white glassy appearance of the DC material was replaced with a soft fleshy overgrowth of cells (FIG. 7A, right). Two-photon examination of normal freshly isolated rat lung allowed visualization of the pattern of normal cell attachment and organization as well as determine the presence of type-I fibrilar collagen (green) (FIG. 7B). After decellularization it was found that the collagen did not maintain its wavy appearance (FIGS. 7C and 7D) and this was also true at least initially in the recellularized lung (FIG. 7E). Elastin was found throughout the DC lung prior to as well as after decellularization (FIG. 7E).

Immunostaining of mESCs grown on DC lung for fourteen days showed production of laminin (FIG. 7G) and collagen-IV (FIG. 7I) by the differentiating mESCs. Both of these components of normal basement membrane were lost during the decellularization process. This was an important result since laminin connects integrins on the basal surface of epithelial cells to the type IV-collagen network of the lamina densa of the basement membrane and forms the framework necessary to support development of lung tissue and proper alveoli formation. FIG. 7I also shows the close association of cytokeratin-18 positive epithelial cells with the newly produced collagen-IV. Pockets of CD140a, platelet derived growth factor receptor-alpha, (PDGFR-α) positive cells were also seen in whole DC lungs after 2 weeks of culture (FIG. 7K). PDGFR-α plays a major role in lung development and is expressed in the mesenchyme of multiple organs during embryonic development. It is also involved in cell proliferation and differentiation of many tissues including the lung. There were also small pockets of pro-SPC positive cells in the distal portions of the DC lung matrix (FIG. 7M) but little indication of complex tissue formation by distal lung cells in tissues isolated after 14 days of culture.

### Example 6

### Complex Tissue Formation

Complex tissue formation is guided by the interplay between stem cells, the extracellular matrix and the cell environment, all of which contribute to the development of complex tissue and eventually to formation of a functional organ. The influence of DC lung on development of three dimensional complex lung tissue equivalents using bioreactor culture of cell-matrix constructs formed from mammalian endogenous lung, mammalian fetal lung or murine embryonic lung stem cells is examined. A rotating bioreactor is used to generate 3D tissue on the DC lung matrix. The use of a rotating bioreactor assures the evaluation of optimal culture conditions (rotational speed of bioreactor, oxygen levels, media exchange times and cell numbers seeded to each decellularized lung). The maximum length of time that tissue constructs can be supported in the bioreactor environment without senescence or necrosis is determined. Ultrastructure of cells within the constructs are examined by confocal, electron microscopy and 2-photon microscopy. There is a down selection for the best cell seeding dose (10⁻³, 10⁻⁵) and selection of growth conditions seen in for production of lung epithelial tissue on the decellularized lung ECM.

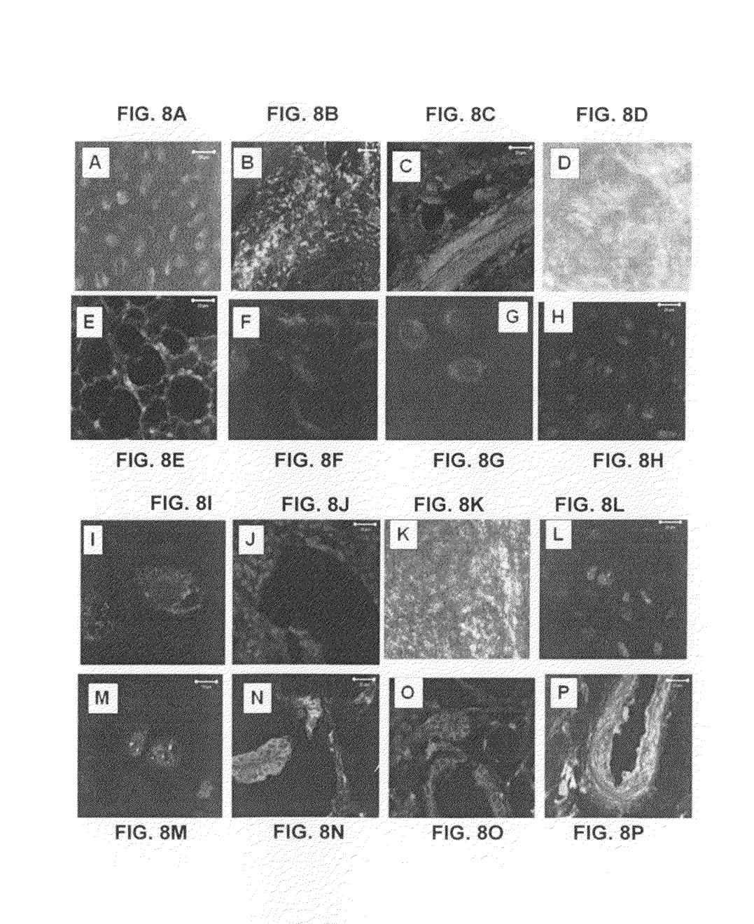

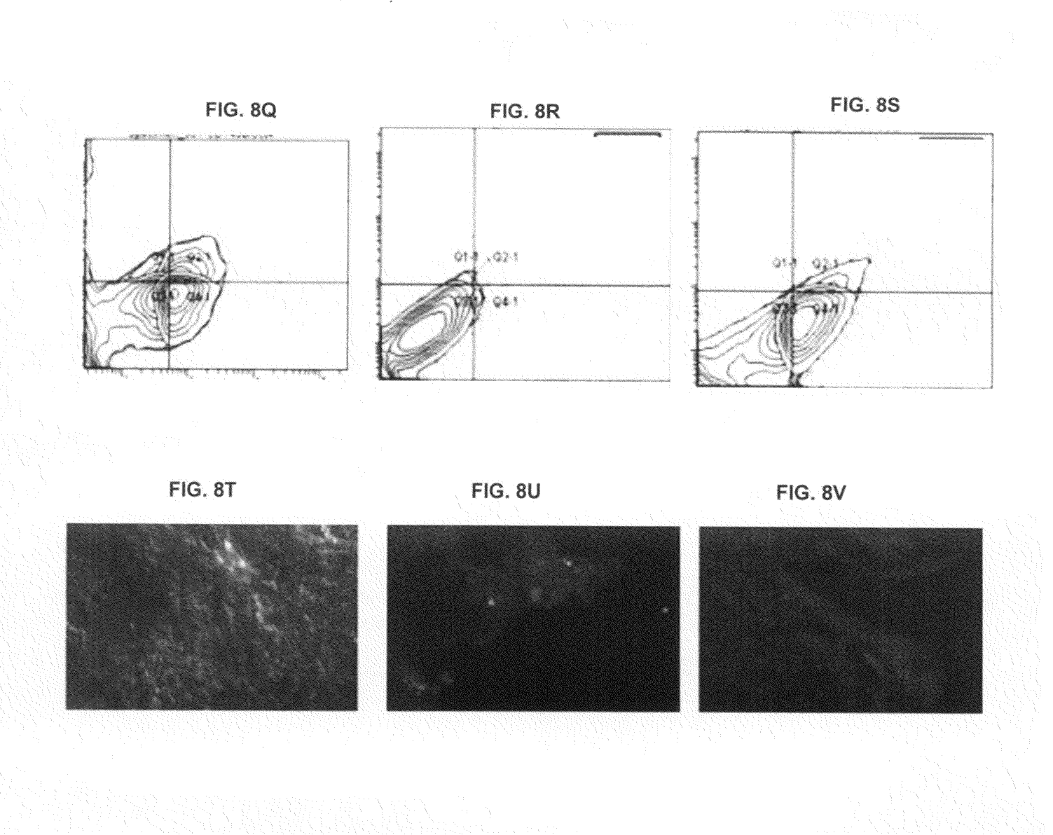

3D tissue formation of DC lung matrix-stem cell constructs is compared to other natural and synthetic matrices which have been used to generate lung endothelium and epithelium to date. FIGS. 8A-8C are stained sections of normal human lung. In the bronchi along the distal airways the main cell types identified were cytokeratin positive CC10+ cells (FIG. 8A). Pro-SPC positive cells were seen in sections of distal lung indicating the presence of type II pneumocytes (FIG. 8B) and cytokeratin positive epithelial cells and CD31 positive endothelial cells (FIG. 8C).

FIGS. 8E-8J show representative sections of tissue formation from Human HLA-1 positive ELPC that were cultured on the rat DC matrix (FIG. 8D-8E). Cells were present that were positive for the endothelial cell marker CD31 (FIG. 8F) as well as the type II pneumocyte marker proSPC (FIG. 8G) in regions of the cultures cell-matrix construct. There were also few areas of the matrix that contained cells positive for the fibroblast marker CD140a (FIG. 8H). The extracellular matrix within the rat lung also supported the production of a few type I, aquaporin-5 positive cells as well as a larger number of CC10 positive Clara cells which were usually seen in large groups or sheets of cells within the DC matrix but were not found by culturing ELPC on any of the other matrices examined. The ELPC were also able to populate the decellularized regions of the trachea quite readily as is seen in FIG. 8K.

Cell nuclei which can be seen by DAPI staining were located within the trachea and were actually embedded within the tracheal ECM. A large number of these cells were shown to express cytokeratin and CC10 (FIGS. 8L-8M) in what had been the areas of the carina and upper regions of the airway. ELPCs exhibited some complex tissue formation as is seen in FIGS. 6N-P where there was an abundance of cytokeratin positive cells (FIG. 8N) and some indications of new collagen production. The cell-matrix construct also contained proSPC positive type II pneumocytes (FIG. 8P). FIG. 6P shows what could be the initial stages of blood vessel formation by CD31 positive (red) endothelial cells which are in close proximity to areas highly positive for laminin. FIGS. 8R-8T are of a representative dot plot of the flow cytometry analysis of the human fetal lung cells used to repopulate the DC lung matrix. Most of the human fetal stem cells were positive for CD 140a or for a fibroblast phenotype. When human fetal stem cells were placed on DC lung they were able to adhere (FIG. 8U) and after culture initial examinations of the engineered tissues showed proSPC (FIG. 8V) staining by very few cells while the majority of cell types seen were cells with a fibroblastic phenotype which expressed CD140a (FIG. 8W).

There were indications of complex tissue formation in the upper lung of 14-day cultures where laminin (FIG. 7G) or collagen-IV (FIG. 7I) were found but only small pockets of differentiated cells were found in regions corresponding to the distal lung.

Lungs cultured for 21 days had a uniform fleshy appearance by sight and by touch that was similar to normal lung. (FIG. 9A). There was no occlusion of the openings of the trachea or bronchi in the recellularized lungs and sheets of cells (FIG. 9B) which were cytokeratin positive lined the upper tracheas (FIG. 9D). In the lower trachea and carina there were patches of CC10 expressing pan-cytokeratin positive cells (FIG. 9F). There were also some regions near the main bronchi where strips of β-actin positive smooth muscle cells were seen (FIG. 9H). In regions of the upper trachea groups of cytokeratin-18 positive ciliated epithelial cells (FIG. 7) and pockets of cells expressing CD31, an endothelial cell marker (FIG. 9M) also were seen.

The cellular composition of the engineered lung changed significantly as one moved from upper trachea and bronchi to more distal lung. In the distal lung there were regions of tissues similar to what would be seen in transitional airways of normal lung in that there were pro-SPC positive type II pneumocytes in hollow epithelial cyst-like structures (FIG. 90) similar to what has been described in 3D culture of mature type II pneumocytes. There were also areas of CD31 positive cells in close proximity to large formations of pan-cytokeratin positive cells in the distal lung (FIG. 9P). These formations (FIG. 9P) were reminiscent of what is seen in vivo during development of vascular tissue in the lung. In what looked like developing transitional airways or brionchio-alveolar junctions, CC10 expressing cells were found next to developing epithelium (FIG. 9R) which were identified by expression of thyroid transcription factor-1 (TTF-1), a transcription factor found in early stages of lung epithelial development.

Finally, production of measurable levels of surfactant protein A by whole lung mESC/AC-lung constructs cultured for 21 days, demonstrated the capability of DC lung to support maturation of fully functional lung cell types which includes the production and secretion of surfactant protein A by Clara cells or Type II pneumocytes (FIG. 9R).

### Discussion

After multiple decellularization attempts, a combination of mechanical, enzymatic and physical processes provided the most efficient and gentle way to remove the cells from the underlying lung ECM without significant loss of collagen or elastin, the major structural components of natural lung. Using confocal and two-photon microscopy to examine sequential sections through DC rat lung it was confirmed that the decellularization process removed cells, cell membranes and the majority of nuclear material. Use of confocal microscopy to identify the presence of DNA in PI or DAPI stained tissues, while not as sensitive a method as gel electrophoresis, did allow for quick routine estimations to monitor changes in DNA in DC lung which allowed for development of better procedures to facilitate DNA removal in our studies. Although trace amounts of DNA were found in the DC lung ECM after the decellularization process the same is true for many commercially available ECM products. Despite the presence of trace amounts of DNA the clinical efficacy and use of these products has been positive.

Multiphoton microscopy of unstained intact sections of the lung provide for evaluation of lung structure and when combined with measurements of second harmonic generation allow for determination of fibrilar collagen-I in lung tissues (17-18).

Two-photon microscopy evaluations of DC rat lung indicated that there did not seem to be a change in pore size or porosity within the decellularized lung tissue and that acellular matrix no longer contained the wavy form of collagen-I seen in natural lung. The decellularization process allowed for retention of the basic lung architecture but unfortunately resulted in the loss of collagen-IV, laminin and fibronectin, all of which are important components of the basement membrane of natural lung ECM.

ECM is secreted and constantly modified by cells as they grow and develop. Changes in ECM structure and composition help to influence cell adhesion and provide critical physical cues that orchestrate tissue formation and function. The composition and physical cues provided by ECM are important for growth of all cell types but are critical for differentiation of ESCs. Composition, concentration and strength of matrix materials such as collagen, fibronectin and laminin have been shown to strongly influence ESC differentiation. Because of this mESC culture was used to examine the capability of DC rat lung to support cell survival, attachment, differentiation and complex tissue formation.

Embryonic stem cells generally have low rates of differentiation to lung lineages (19-20) although one study reported that 24% of mESCs had been differentiated to surfactant protein C producing cells in aggregate cocultures of mESCs and fetal lung cells (21-22).

In order to support the recellularization process a “lung environment” was created by culturing mESC/matrix constructs with a combination of soluble lung specific growth factors supplemented with mature lung homogenate. A rotary bioreactor was used to create a supportive fluid environment for engineered tissue constructs similar to what one would see in vivo for fetal development during organogenesis. Rotary bioreactor culture also reduces sheer stress and maintains a constant flow of nutrients to the developing tissue.

Normal cells probe, pull and push at their surrounding matrix and the forces generated by these interactions of cells with ECM influence both the response of individual cells and the subsequent development of tissues. Gelfoam is a gelatin based sponge, made predominantly of denatured type-I collagen, and is the simplest of the matrices we examined. The collagen-I PF-127 hydrogel matrix used in this study is also simple matrix and only has one component of normal lung ECM, collagen-I, immersed within the PF-127 soft hydrogel. Matrigel contains the basement membrane components collagen-IV and laminin and small amounts of other ECM materials, making it more complex than the previously mentioned matrix materials. DC rat lung contains collagen-1, elastin and small amounts of other ECM. Matrigel and DC lung, due to fibrosity alone, were fairly close in terms of matrix stiffness. In the assessment of 4 um sections of each of these matrices (seen in FIGS. 6A, 6C, 6E and 6G) was categorized according to degree of porosity content where Gelfoam<Collagen-I/PF-127<Matrigel<DC lung. In terms of fibrosity or stiffness as estimated by fiber content and ECM components, the categorization was according to degree of stiffness where Gelfoam<Collagen-UPF-127<Matrigels≦DC lung. Even within the lung there are regional differences in ECM composition, fibrosity and porosity. When comparing the fibrosity and porosity of regions within whole lung it was obvious that upper lung and trachea were much more fibrous and had less porosity than distal lung. Upper lung had increased stiffness (fibrosity) compared to distal lung and supported the differentiation of mESC towards lineages of cells that are normally found in upper lung and trachea such as Clara cells and cytokeratin positive epithelial cells. In these studies Clara cells and ciliated epithelial cells were only seen in upper lung and pro-SPC positive cells were only seen in regions of distal lung.

After 7 days the phenotype of mESC grown in plate culture, on DC rat lung or on Gelfoam, Matrigel and collagen-I/PF-127 matrices was analyzed. Apoptosis induction was studied since programmed cell death or apoptosis is known to play an important role in embryo development, contributing to the appropriate formation of various organs and structures. Surprisingly, plate or 2D culture always had the highest levels of apoptosis and was the least efficient system to induce production of differentiation-associated proteins specific to epidermal or endodermal lineages compared to any of the 3D matrix cultures, although the same numbers of cells and differentiation medium was used. DC lung constructs retained cells better, enhanced cell survival and induced higher levels of lung specific lineages than did collagen-I/PF127, Matrigel, or Gelfoam matrices. An explanation for this observation might be that even with the ECM changes seen as a result of the decellularization protocol, simple matrices such as Matrigel, Gelfoam or collagen-I/PF-127 do not provide the requisite physical cues or mechanical influences necessary to support good lung site-specific differentiation of cells. This is consistent with the studies that support the premise that local growth rate of tissue formed by cells is influenced by the geometry as well as the consistency and structure of the ECM (22-26).

To determine if DC lung matrix could influence mESC 3D tissue development, tissue development was examined in whole DC rat lung constructs cultured in a rotary bioreactor for 14 days or 21 days. By 14 days of culture, cellular structures formed by the differentiating mESC had secreted extracellular matrix components lacking in DC lung including both collagen-IV and laminin as demonstrated by immunostaining. Areas were observed where groups of cells expressed CD140a or PDGFR-α. This was an important result because PDGFR-α has been shown to be crucial for alveolar myofibroblast ontogeny and alveogenesis. Expression of PDGFR-α in these cultures was a good indication that mESC differentiation towards lung lineages and formation of nascent ECM by developing myofibroblasts was in process.

Type II alveolar epithelial cells (TYII AEC) are the first type of alveolar epithelial cell (AEC) to form in human and rodents during normal fetal lung development. Although there was differentiation of cells to endodermal lineages by day 14, there was no extensive 3D complex tissue formation or extensive production of TY II AECs. By 21 days it was hoped to see complex tissue formation in whole lung cultures since the normal gestation period for C57BL 6 mice is 19-22 days. In 21-day lung cultures site-specific differentiation of mESCs was evidenced by production of discrete regions of β-actin expressing smooth muscle cells, cytokeratin-18 as well as CC10 expressing cells in regions that included both the trachea and bronchi (regions 1 and 2 of FIG. 9A). Along the inner surfaces of the trachea and bronchi we were able to detect sheets of cells that expressed epithelial cell markers such as TTF-1, cytokeratin-18 or CC10 which also included a few ciliated cells. An extensive large scale development of ciliated epithelial cells did not occur because the use of the bioreactor for cell culture did not provide an adequate air interface to the culture which has been shown to be necessary to induce maturation and development of ciliated epithelia (27).

In fetal lung development, alveolar epithelial cell differentiation begins during the canalicular stage of development however true alveoli formation does not occur until much closer to birth. In 21-day cultures immunohistochemical staining showed formation of sheets of TTF-1 expressing immature epithelial cells and cyst-like formations of pro-SPC expressing TY II AEC in the distal lung. Other indications of epithelial cell differentiation include the secretion of the major lung surfactant protein, surfactant protein A (SPA). In the mouse SPA is first detected in amniotic fluid at 17 days post coitum and amounts of SPA rise progressively to term (19-21 days post coitum). Secretion of measurable amounts of surfactant protein A by TY II AEC or Clara cells was seen in 21-day cultures indicating that maturation of the lung tissues was taking place although not at the same rate as occurs in utero.

A vascular supply is critical to the formation of good functional tissues and because of this it was evaluated whether mESCs were able to differentiate into endothelial lineages and organize into vessels in the engineered tissues. Immunochemical staining for CD31 showed that DC rat lung supported both higher levels of endothelial cell differentiation and, in whole-lung cultures, the formation of very simple capillary-like networks.

These data suggest that acellular lung retains enough scaffold-mediated biological signals to direct mESC toward lung-specific lineages and to guide lung specific tissue development in vitro. This data also demonstrates the ability of ECM cues to influence mESC differentiation in a site-specific manner. In Summary DC natural lung allowed for better retention of cells with more differentiation of mESCs into epithelial and endothelial lineages than was seen in matrices previously used to generate engineered lung. In constructs formed from mESCs and whole DC lung there were indications of differentiating ESC organized into three-dimensional (3D) structures reminiscent of complex tissues. In whole DC lung/mESC constructs, there was expression of Thyroid Transcription Factor-1 (TTF-1) an immature lung epithelial cell marker, Pro-surfactant protein C (pro-SPC) a type II pneumocyte marker, PECAM-1/CD31 an endothelial cell marker, CD140A or PDGFR-α which is expressed in mesenchyme during embryonic development, and Clara Cell Protein 10 (CC10) a Clara Cell marker, as well as development of a few discrete pockets of ciliated epithelial cells. There was also evidence of site specific differentiation in the trachea and distal lung. In the trachea there were formation of sheets of cytokeratin-18 positive cells and CC10 expressing Clara cells, near the carina and upper bronchi. Type II pneumocytes found in the distal lung formed hollow alveolar-like cysts lined by a monolayer of epithelial cells, which produced both pro-SPC, the nonsecreted form of surfactant protein C, as well as production of SPA.

The following references were cited herein:
1. Murray et al. Lancet, 1997, 349:1498-1504
2. MMWR Weekly, 2008, 57 (45):1229-1232b
3. Foster et al. COPD, December 2006, 3 (4) 177-178
4. Sugihara et al. Am J Pathol, 1993, 142:783-792
5. Cortiella et al. Tissue Eng, 2006, 12:1213-1225
6. Mondrinos et al. Am J Physiol Lung Cell Mol Physiol, 2007, 292:L510-518
7. Chen et al. Tissue Eng, 2005, 11:1436-1448
8. Nichols et al. Proceedings of the American Thoracic Society 5, 723, 2008.
9. Nichols et al. Organogenesis 5 (2), 57, 2009.
10. Ott et al. Nat Med, 2008, 14:213-221
11. Gilbert et al. Biomaterials, 2006, 27:3675-3683
12. Macchiarini et al. Lancet, 2008, 372:2023-2030
13. Badylak S F. Seminars in Cell and Developmental Biology 13(5), 377, 2002.
14. Badylak et al. Acta Biomaterialia 5, 1, 2009.
15. Gilbert et al. Biomaterials 27, 3675, 2006.
16. Sellaro et al. Tissue Engineering 13 (9), 2301, 2007.
17. Agarwal et al. Tissue Engineering 7, 191, 2001.
18. Pena et al. Microscopy Research and Technique 70, 162, 2007.
19. Rippon et al. Proceedings of the American Thoracic Society 5, 717, 2008.
20. Samadikuchaksaraei et al. Tissue Engineering; 12 (4), 867, 2006.
21. Denham et al. Am. J. of Physiol. Lung Cellular & Molecular Physiology 292, L1241, 2007.
22. Engler et al. Cell 126, 677, 2006.
23. Engler et al. Journal of Musculoskeletal and Neuronal Interactions 7, 335, 2007.
24. Vogel et al. Nature Reviews. Molecular Cell Biology 7, 265, 2006.
25. McBeath et al. Developmental Cell 6, 483, 2004.
26. Shannon et al. Biochimica et Biophysica Acta 931, 143, 1987.
27. Gray et al. Am J Respir Cell Mol Biol 14: 104, 1996.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

What is claimed is:

1. A method for producing decellularized lung extracellular matrix, comprising:
inducing cellular damage to native lung tissue; and
removing cellular debris produced by the cellular damage to the lung tissue, wherein remaining tissue is the decellularized lung extracellular matrix.

2. The method of claim 1, wherein the step of inducing cellular damage comprises alternating cycles of rapid freezing and rapid thawing of native lung tissue or sonicating the native lung tissue.

3. The method of claim 1, wherein the step of removing cellular debris comprises contacting the damaged lung tissue with a detergent or with peracetic acid within a continuously rotating bioreactor.

4. The method of claim 3, wherein the detergent is 1% SDS continually circulated within the rotating bioreactor for about 5 weeks.

5. The method of claim 4, wherein the step of removing cellular debris further comprises treating any remaining damaged or intact cells with DNAase and RNAase.

6. The method of claim 1, further comprising:
seeding onto or into the DC lung ECM one or both of endogenous progenitor lung cells or stem cells to produce a cell:matrix construct.

7. The method of claim 6, further comprising:
culturing the cell:matrix construct in vitro in a bioreactor under conditions effective to induce differentiation and growth of the cells toward a lung lineage within the matrix thereby producing functional lung tissue

8. The method of claim 7, further comprising:
implanting the functional lung tissue at one or more non-functioning sites of interest within a lung to restore at least some function thereto.

9. The method of claim 1, wherein the native lung tissue comprises mammalian trachea and lungs.

10. A method for producing engineered functional lung tissue, comprising:
decellularizing native lung tissue to produce a decellularized lung extracellular matrix (DC lung);
isolating one or both of endogenous progenitor lung cells or stem cells;
seeding onto or into the DC lung the endogenous progenitor lung cells or stem cells to produce a cell:matrix construct; and
culturing the cell:matrix construct in a bioreactor under conditions effective to induce differentiation and growth of the cells toward a lung lineage within the matrix thereby producing the engineered functional lung tissue.

11. The method of claim 10, further comprising implanting the engineered lung tissue into a subject having a pulmonary disease, a pulmonary disorder or an injury to pulmonary tissue.

12. The method of claim 10, wherein the engineered lung tissue comprises cell types and cell numbers corresponding to native lung tissue.

13. The method of claim 12, wherein the cell types are type 1 epithelial cells, type 2 epithelial cells or endothelial cells

14. A composition comprising decellularized lung extracellular matrix and one or both of endogenous progenitor lung cells or stem cells seeded thereon or therein.

15. The composition of claim 14, wherein the mammalian lung is a human lung.

16. An implantable composition comprising decellularized lung extracellular matrix and engineered functional lung tissue differentiated from endogenous lung progenitor cells grown in or on the decellularized extracellular matrix.

17. The implantable composition of claim 16, wherein the endogenous lung progenitor cells are human lung progenitor cells

18. A method for treating a lung to restore function thereto in a subject in need of such treatment, comprising:
implanting into the lung of the subject the composition of claim 16, wherein growth of the lung tissue comprising the implantable composition restores at least partial function to the lung.

19. The method of claim 18, wherein the subject has a pulmonary disease, a pulmonary disorder or an injury or damage to pulmonary tissue.
